(19) United States
(12) Patent Application Publication
(10) Pub. No.: US 2026/0090896 A1
(43) Pub. Date: Apr. 2, 2026

(54) IMPLANT SYSTEMS AND METHODS FOR STABILIZING VERTEBRAL BODIES FROM A POSTERIOR AND POSTERIOR LATERAL APPROACH TO THE SPINE

(71) Applicant: Foundation Surgical Group, Inc., Scottsdale, AZ (US)

(72) Inventors: Randal R. Betz, Bradenton, FL (US); Lawrence G. Lenke, New York, NY (US); Michael Sherman, Memphis, TN (US); Charlie Barfield, Nesbit, MS (US); Dimitri K. Protopsaltis, Memphis, TN (US); Asher Breverman, Scottsdale, AZ (US); Christopher Reah, Taunton (GB)

(73) Assignee: Foundation Surgical Group, Inc., Scottsdale, AZ (US)

(21) Appl. No.: 19/322,646

(22) Filed: Sep. 8, 2025

Related U.S. Application Data
(63) Continuation of application No. PCT/US25/45275, filed on Sep. 7, 2025.
(63) Continuation-in-part of application No. PCT/US25/45274, filed on Sep. 7, 2025.
(60) Provisional application No. 63/692,618, filed on Sep. 9, 2024.
(60) Provisional application No. 63/692,693, filed on Sep. 9, 2024.
(60) Provisional application No. 63/692,618, filed on Sep. 9, 2024.
(60) Provisional application No. 63/692,693, filed on Sep. 9, 2024.
(60) Provisional application No. 63/692,618, filed on Sep. 9, 2024.
(60) Provisional application No. 63/692,693, filed on Sep. 9, 2024.

(51) Int. Cl. A61F 2/44; A61B 17/56; A61F 2/30

(52) U.S. Cl. CPC A61F 2/447; A61B 17/56; A61F 2/30749; A61B 2017/564; A61F 2002/30261; A61F 2002/30367; A61F 2002/30372; A61F 2002/30622

(57) ABSTRACT

An implant system comprising at least one staple, a cage and a coupling mechanism is disclosed. The implant system is secured to the bone by moving the staple with the coupling mechanism whereby the staple frictionally and mechanically engages or embeds itself in the bone. In some embodiments, the coupling mechanism includes a keyway mechanism to constrain the movement of the staple. In some embodiments the staple comprises two staples. The implant system may be configured for use as an interbody or intrabody implant system. In some embodiments, the cage comprises multiple cage sections. Some embodiments of the implant system are configured to be positioned from one of a posterior approach trajectory, a posterior lateral approach trajectory, or a posterior oblique approach trajectory. Some embodiments of the implant system are configured for use in a posterior lumbar interbody fusion (PLIF) or transforaminal lumbar interbody fusion (TLIF) procedure.

### CROSS-REFERENCE TO RELATED APPLICATIONS

This application claims benefit to U.S. Pat. App. No. 63/692,618, filed Sep. 9, 2024 and claims benefit to U.S. Pat. App. No. 63/692,693, filed Sep. 9, 2024; this application is a continuation of PCT App. No. PCT/US25/45275, filed Sep. 7, 2025; this application is a continuation in part of PCT App. No. PCT/US25/45274, filed Sep. 7, 2025; PCT App. No. PCT/US25/45275 claims benefit to U.S. Pat. App. No. 63/692,693; PCT App. No. PCT/US25/45275 claims benefit to U.S. Pat. App. No. 63/692,618; PCT App. No. PCT/US25/45274 claims benefit to U.S. Pat. App. No. 63/692,693; PCT App. No. PCT/US25/45274 claims benefit to U.S. Pat. App. No. 63/692,618; the entire contents of which are incorporated herein by reference.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISC APPENDIX

Not applicable.

### BACKGROUND OF THE INVENTION

Aspects of the present disclosure relate to orthopedic implants. Embodiments of the implants may be used as spinal implants to stabilize vertebra from a posterior, posterior lateral, PLIF and TLIF approach trajectories. Embodiments of the implants may be configured to augment the vertebral body or fuse multiple vertebral bodies to decompress neural elements and alter the alignment of the spine.

### BRIEF SUMMARY OF THE INVENTION

The systems, methods, and devices of the disclosure each have several aspects, no single one of which is solely responsible for its desirable attributes. Without limiting the scope of this disclosure as expressed by the claims which follow, some features will now be discussed briefly. After considering this discussion, and particularly after reading the section entitled “Detailed Description” one will understand how the features of this disclosure provide advantages that include improved communications between access points and stations in a wireless network.

The following summary is included only to introduce some concepts discussed in the Detailed Description below. This summary is not comprehensive and is not intended to delineate the scope of protectable subject matter, which is set forth by the claims presented at the end.

Within this description, the terms far, distal, and contralateral are used interchangeably and are intended to be interpreted as defining that one thing is distant from another such as distance from a point of origin, situated away from a point of origin, and pertaining to the other side. Also, the terms near, proximal and ipsilateral are used interchangeably within this description and are intended to be interpreted as defining a short distance away from another such as away from a point of origin, situated toward a point of origin and belonging to or occurring on the same side of a body.

In some of the disclosed embodiments of an implant system having a cage and a staple, the staple is moveable relative to the cage. The movability of the staple relative to the cage provides several features to the implant system. This movable configuration allows portions of the staple to move longitudinally in and out, towards and away from the cage. This allows for better control of the alignment, location and positioning of the staple shaft, and when the staple shaft is operably coupled to the staple head in a way that allows the staple head to rotate with the staple shaft, this allows for better control of the alignment and location and positioning of the staple head. This control allows the staple head to be moved through alignments and locations and positions that better accommodate the surface of the bone to better secure the staple and implant device to the bone.

In some of the disclosed embodiments of an implant system, the implant system is secured to a bone with a compressive force. The ability of the implant system to secure the implant device with a compression force from opposite sidewalls of a bone provides a more secure anchoring of the implant device to the bone as compared to anchoring from one side of the bone. The orientation of the implant device when implanted laterally also provides a lateral platform on the device to anchor additional devices such as tether screws, tulip head screws and rods or tethers or cords to the implant device. The orientation of the implant device when implanted laterally also provides the ability for the implant to be implanted from orientations that take advantage of the surgical benefits of approach trajectory orientations such as lateral or oblique.

In one aspect, the present disclosure provides an orthopedic implant device comprising a cage, a first staple, a second staple and the first staple and the second staple are movable relative to the cage whereby the first staple and the second staple are each configured to move from an insertion position to a stabilized position.

In some embodiments, the implant device is configured to be positioned from one of a posterior trajectory, a posterior lateral trajectory, or a posterior oblique trajectory. In some embodiments, the implant device is configured to be used in a posterior lumbar interbody fusion (PLIF) procedure or a transforaminal lumbar interbody fusion (TLIF) procedure.

In one aspect, the present disclosure provides an orthopedic implant device comprising: a cage; a staple comprising a staple shaft; a coupling mechanism comprising a coupling element configured to engage the staple shaft where the coupling element is configured to influence a movement of the staple shaft; and the coupling mechanism comprising a keyway mechanism configured to constrain the movement of the staple shaft.

Embodiments may include one or more of the following features. The orthopedic implant device where the movement of the staple shaft comprises a rotation of the staple shaft and a translation of the staple shaft; the coupling mechanism is configured to influence the rotation of the staple shaft and the translation of the staple shaft; and the coupling mechanism is configured to constrain the rotation of the staple shaft and the translation of the staple shaft. The orthopedic implant device where the keyway mechanism comprises: a keyway and a key configured to engage the keyway to constrain the movement of the staple shaft. The orthopedic implant device where the movement of the staple shaft is a rotation of the staple shaft and the key is configured to engage the keyway to constrain the rotation of the staple shaft. The orthopedic implant device where the implant device is configured to be implanted from a posterior approach trajectory. The orthopedic implant device where the implant device further comprises a second staple. The orthopedic implant device where the second staple comprises: a second staple head and a second staple shaft; the second staple head comprising a first portion and a second portion; and the second staple head configured to rotate about the second staple shaft where the first portion extends in a non-parallel orientation above an upper surface of the cage and the second portion extends in a non-parallel orientation below a lower surface of the cage. The orthopedic implant device where the implant device is configured to be implanted from a posterior approach trajectory. The orthopedic implant device where the second staple comprises a second staple shaft and the coupling mechanism further comprises an engagement mechanism configured to influence a movement of the second staple shaft with the movement of the staple shaft. The orthopedic implant device where the movement of the second staple shaft comprises a rotation of the second staple shaft and the coupling mechanism comprises an engagement portion of the staple shaft configured to engage an engagement portion of the second staple shaft where a rotation movement of the staple shaft influences a rotation movement of the second staple shaft. The orthopedic implant device where: the engagement portion of the staple shaft comprises an exterior surface profile of the staple shaft; and the engagement portion of the second staple shaft comprises a mating surface profile of the second staple shaft where when the engagement portion of the staple shaft is received in the engagement portion of the second staple shaft, the rotation movement of the staple shaft influences the rotation movement of the second staple shaft. The orthopedic implant device where the implant device further comprises a plate. The orthopedic implant device where the plate comprises: a rigid element having a first portion and a second portion; the first portion configured to extend above the cage; and a second portion configured to extend below the cage. The orthopedic implant device where the implant device is configured to be implanted from a posterior approach trajectory. The orthopedic implant device where the movement of the staple shaft is a translation of the staple shaft and the key is configured to engage the keyway to constrain the translation of the staple shaft. The orthopedic implant device where: the movement of the staple shaft comprises a rotation of the staple shaft and a translation of the staple shaft; the key comprises a flexible prong coupled to the staple shaft; the key having a rotation constraining portion configured to engage the keyway and constrain the rotation of the staple shaft; the key having a rotation allowing portion configured to engage the keyway and allow the rotation of the staple shaft; and the key comprises a translation stop portion configured to engage the keyway and constrain the translation of the staple shaft. The orthopedic implant device where: the movement of the staple shaft comprises a rotation of the staple shaft and a translation of the staple shaft; the keyway comprises a through-hole in the cage; the through-hole having a rotation constraining portion configured to engage the key and constrain the rotation of the staple shaft; and the through-hole having a deployment position portion configured to engage the key and constrain the rotation of the staple shaft. The orthopedic implant device where: the movement of the staple shaft is a rotation of the staple shaft and a translation of the staple shaft; the key comprises a flexible prong coupled to the staple shaft: the key having a rotation constraining portion, a rotation allowing portion and a translation stop portion; the keyway comprises a through-hole in the cage; the through-hole having a rotation constraining portion and a deployment position portion; the rotation constraining portion of the key configured to engage the rotation constraining portion of the through-hole where the translation of the staple shaft is allowed but the rotation of the staple shaft is constrained; the translation stop portion of the key configured to engage the through-hole where the translation of the staple shaft is stopped; the rotation allowing portion of the key configured to engage the rotation constraining portion of the through-hole where the rotation of the staple shaft is allowed; and the rotation allowing portion of the key configured to engage the deployment position portion of the through-hole where the rotation of the staple shaft is constrained in a deployment position. The orthopedic implant device where: the staple comprises the staple shaft and a staple head; a second staple head and a second staple shaft; the staple head comprising a first portion and a second portion; and the staple head configured to rotate about the staple shaft where the first portion extends in a non-parallel orientation above an upper surface of the cage and the second portion extends in a non-parallel orientation below a lower surface of the cage.

In one aspect, the present disclosure provides an orthopedic implant device comprising: a cage having a proximal end and a distal end; one or more staple positioned near the distal end of the cage; the one or more staple configured to move relative to the cage; and where the one or more staple is configured to move relative to the cage from an insertion position to an extended position.

Embodiments may include one or more of the following features. The orthopedic implant device where the orthopedic implant device has a drive coupler configured to rotate about a drive coupler rotation axis and the one or more staple is configured to move to the extended position along one or more staple longitudinal axis that is non-parallel to the drive coupler rotation axis. The orthopedic implant device where the implant device is configured to be secured to a vertebral body in an interbody fusion procedure comprising one of a posterior lumbar interbody fusion (PLIF) procedure or a transforaminal lumbar interbody fusion (TLIF) procedure. The orthopedic implant device where: the one or more staple comprises a first staple and a second staple; the first staple having a first staple head positioned near the distal end of the cage; and the second staple having a second staple head positioned near the proximal end of the cage. The orthopedic implant device where the first staple and the second staple are coupled where the first staple and the second staple are configured to rotate together. The orthopedic implant device where the first staple head and the second staple head are each positioned within an outer wall of the cage. The orthopedic implant device where the first staple head and the second staple head are each positioned within an outer wall of the cage and the first staple head and the second staple head are configured to engage a vertebral body within the outer wall of the vertebral body when the orthopedic implant device is in a deployed position. The orthopedic implant device where the first staple head and the second staple head are each positioned outside of an outer wall of the cage. The orthopedic implant device where the first staple head and the second staple head are each positioned outside of an outer wall of the cage and the first staple head and the second staple head are configured to engage the outer wall of a vertebral body when the orthopedic implant device is in a stabilized position. The orthopedic implant device may include a keyway mechanism configured to influence a movement of the staple. The orthopedic implant device where the keyway mechanism comprises a keyway configured to influence the movement of the staple and a key configured to engage the keyway to constrain the movement of the staple. The orthopedic implant device where: the movement of the one or more staple is one or more rotation of the one or more staple; the keyway is configured to constrain the rotation of the one or more staple; and the key is configured to constrain the rotation of the one or more staple. The orthopedic implant device where: the movement of the one or more staple is a translation of the one or more staple; the keyway is configured to constrain the translation of the one or more staple; and the key is configured to constrain the translation of the one or more staple. The orthopedic implant device where: the key comprises a flexible prong coupled to the one or more staple: and the key having a rotation constraining portion, a rotation allowing portion and a translation stop portion. The orthopedic implant device where the keyway comprises a through-hole in the cage and the through-hole have a rotation constraining portion and a deployment position portion. The orthopedic implant device where: the one or more staple comprises one or more staple shaft and one or more staple head; the movement of the one or more staple shaft is a rotation of the one or more staple shaft and a translation of the one or more staple shaft; the key comprises a flexible prong coupled to the one or more staple shaft: the key having a rotation constraining portion, a rotation allowing portion and a translation stop portion; the keyway comprises a through-hole in the cage; the through-hole having a rotation constraining portion and a deployment position portion; the rotation constraining portion of the key configured to engage the rotation constraining portion of the through-hole where the translation of the one or more staple shaft is allowed but the rotation of the one or more staple shaft is constrained; the translation stop portion of the key configured to engage the through-hole where the translation of the one or more staple shaft is stopped; the rotation allowing portion of the key configured to engage the rotation constraining portion of the through-hole where the rotation of the one or more staple shaft is allowed; and the rotation allowing portion of the key configured to engage the deployment position portion of the through-hole where the rotation of the one or more staple shaft is constrained in a deployment position. The orthopedic implant device where: the cage comprises a first cage section and a second cage section; the one or more staple comprises a first staple and a second staple; the first staple coupled to the first cage section and the second staple coupled to the second cage section; the first staple having a first staple head positioned near a distal end of the first cage section; and the second staple having a second staple head positioned near a proximal end of the second cage section. The orthopedic implant device where: the orthopedic implant device having a drive coupler configured to rotate about a drive coupler rotation axis; the one or more staple is configured to move to a deployed position; and the one or more staple is configured to rotate about one or more staple rotation axis that is non-parallel to the drive coupler rotation axis to the deployed position. The orthopedic implant where: the one or more staple comprises one or more staple head having a first staple portion and a second staple portion; and the one or more staple is configured to move to a deployed position where the first staple portion is configured to extend above the cage and engage a first bone positioned above the cage and the second staple portion is configured to extend below the cage and engage a second bone positioned above the cage. The orthopedic implant device where: the cage having a posterior side and an anterior side on either side of a longitudinal midline of the cage; the posterior side of the cage is configured to be positioned in a posterior orientation to a mammalian body when the orthopedic implant device is implanted in the mammalian body; the anterior side of the cage is configured to be positioned in an anterior orientation to the mammalian body when the orthopedic implant device is implanted in the mammalian body; the one or more staple is positioned near the distal end of the cage; a drive coupler positioned on the posterior side of the cage; and the drive coupler is configured to be manipulated from the posterior side of the cage and move the one or more staple to the extended position. The orthopedic implant device where: the orthopedic implant device is configured to be inserted towards a vertebral body from an approach trajectory; and the one or more staple is configured to be movable about one or more axis that is non-parallel to the approach trajectory of the orthopedic implant device. The orthopedic implant device where: the orthopedic implant device is configured to be inserted towards a vertebral body from an approach trajectory; and the one or more staple is configured to rotate about one or more staple rotation axis that is non-parallel to the approach trajectory of the orthopedic implant device. The orthopedic implant device where: the orthopedic implant device is configured to be inserted towards a vertebral body from an approach trajectory; and the one or more staple is configured to translate along one or more staple translation axis that is non-parallel to the approach trajectory of the orthopedic implant device. The orthopedic implant device where: the orthopedic implant device is configured to be inserted towards a vertebral body from an approach trajectory; and the approach trajectory of the implant device is one of a posterior approach trajectory, a posterior lateral approach trajectory or a posterior oblique approach trajectory.

In one aspect, the present disclosure provides an orthopedic implant device comprising: a cage comprising a first cage section and a second cage section; the first cage section having a first staple; the second cage section having a second staple; the first staple is movable relative to the first cage section where the first staple is configured to move from an insertion position to a deployed position; and the second staple is movable relative to the second cage section where the second staple is configured to move from an insertion position to a deployed position.

Embodiments may include one or more of the following features. The orthopedic implant device where: the cage having a proximal end and a distal end; each of the first cage section and the second cage section having a longitudinal midline and a posterior side and an anterior side on either side of the longitudinal midline; the posterior side of the cage is configured to be positioned in a posterior orientation to a mammalian body when the orthopedic implant device is implanted in the mammalian body; the anterior side of the cage is configured to be positioned in an anterior orientation to the mammalian body when the orthopedic implant device is implanted in the mammalian body; a first drive coupler positioned on the posterior side of the first cage section; a second drive coupler positioned on the posterior side of the second cage section; the first drive coupler configured to be manipulated from the posterior side of the first cage section and move the first staple to the deployed position; and the second drive coupler configured to be manipulated from the posterior side of the second cage section and move the second staple to the deployed position. The orthopedic implant device where: the first drive coupler is configured to move the first staple to an extended position; and the second drive coupler is configured to move the second staple to an extended position. The orthopedic implant where: the first drive coupler is configured to move the first staple to a stabilized position; and the second drive coupler is configured to move the second staple to a stabilized position. The orthopedic implant device where: the first drive coupler is configured to rotate about a first drive coupler rotation axis; the first staple is configured to move along a first staple longitudinal axis that is non-parallel to the first drive coupler rotation axis; the second drive coupler is configured to rotate about a second drive coupler rotation axis; and the second staple is configured to move along a second staple longitudinal axis that is non-parallel to the second drive coupler rotation axis. The orthopedic implant device where: the first drive coupler is configured to rotate about a first drive coupler rotation axis; the first staple is configured to rotate about a first staple rotation axis that is non-parallel to the first drive coupler rotation axis; the second drive coupler is configured to rotate about a second drive coupler rotation axis; and the second staple is configured to rotate about a second staple rotation axis that is non-parallel to the second drive coupler rotation axis.

In one aspect, the present disclosure provides a method to secure an implant device to a vertebral body, the method comprising: performing an osteotomy through a vertebral body to create an osteotomy space, providing an implant device comprising a cage and one or more staple, positioning the cage in the osteotomy space created within the vertebral body, and positioning the one or more staple to engage one or more side wall of the vertebral body whereby the one or more staple secures the implant device to the vertebral body.

In one aspect, the present disclosure provides a method to secure an implant device to a vertebral body, the method comprising: providing access to a vertebral body from an access portal positioned posterior to the vertebral body; performing an osteotomy through the vertebral body to create an osteotomy space between a first bone portion and a second bone portion; providing an implant device comprising a cage and one or more staple; positioning the implant device in the osteotomy space created within the vertebral body from a posterior approach trajectory; the cage movably coupled to the one or more staple; and moving the one or more staple to engage the first bone portion and the second bone portion of the vertebral body where the one or more staple secures the implant device to the first bone portion and the second bone portion of the vertebral body.

Embodiments may include one or more of the following features. The method where: the cage movably coupled to the one or more staple with a coupling mechanism; the coupling mechanism comprising a drive coupler configured to move the one or more staple; the drive coupler configured to be engaged by a drive rod from the access portal positioned posterior to the vertebral body; and the step of moving the one or more staple to engage the first bone portion and the second bone portion of the vertebral body comprises: engaging the drive coupler with the drive rod to move the one or more staple to a deployed position, and engaging the drive coupler with the drive rod to move the one or more staple from the deployed position to a stabilized position where the cage is secured to the first bone portion and the second bone portion of the vertebral body. The method where: the one or more staple comprises a first staple and a second staple; the first staple having a first staple head positioned proximal to a distal end of the cage; and the second staple having a second staple head positioned proximal to a proximal end of the cage; the first staple head and the second staple head are each positioned outside of an outer wall of the cage; and the first staple head and the second staple head are configured to engage an outer wall of both the first bone portion and the second bone portion of the vertebral body when the first staple and the second staple are in a stabilized position. The method where: the one or more staple comprises a first staple and a second staple; the first staple having a first staple head positioned proximal to a distal end of the cage; and the second staple having a second staple head positioned proximal to a proximal end of the cage; the first staple head and the second staple head are each positioned within an outer wall of the cage; and the first staple head and the second staple head are configured to engage both the first bone portion and the second bone portion of the vertebral body when the first staple and the second staple are in a stabilized position. The method where: the one or more staple comprises a first staple and a second staple; the cage comprises a first cage section coupled to the first staple and a second cage section coupled to the second staple; the first staple having a first staple head positioned proximal to a distal end of the cage where the first staple head engages both the first bone portion and the second bone portion when the implant device is in a stabilized position; and the second staple having a second staple head positioned proximal to a proximal end of the cage where the second staple head engages both the first bone portion and the second bone portion when the implant device is in a stabilized position.

In one aspect, the present disclosure provides a method to secure an implant device to a first vertebral body and a second vertebral body, the method comprising: providing access to a first vertebral body and a second vertebral body from an access portal positioned posterior to the first vertebral body and the second vertebral body; accessing a space between a first bone portion of the first vertebral body and a second bone portion of the second vertebral body; providing an implant device comprising a cage and one or more staple; the cage movably coupled to the one or more staple; positioning the implant device in the space between the first bone portion and the second bone portion; and moving the one or more staple to engage the first bone portion and the second bone portion where the one or more staple secures the implant device to the first bone portion and the second bone portion.

Embodiments may include one or more of the following features. The method where: the cage movably coupled to the one or more staple with a coupling mechanism; the coupling mechanism comprising a drive coupler configured to move the one or more staple; the drive coupler configured to be engaged by a drive rod from the access portal positioned posterior to the vertebral body; and the step of moving the one or more staple to engage the first bone portion and the second bone portion of the vertebral body comprises: engaging the drive coupler with the drive rod to move the one or more staple to a deployed position, and engaging the drive coupler with the drive rod to move the one or more staple from the deployed position to a stabilized position where the cage is secured to the first bone portion and the second bone portion. The method where: the one or more staple comprises a first staple and a second staple; the first staple having a first staple head positioned near a distal end of the cage; and the second staple having a second staple head positioned near a proximal end of the cage; the first staple head and the second staple head are each positioned outside of an outer wall of the cage; and the first staple head and the second staple head are configured to engage an outer wall of both the first bone portion and the second bone portion when the implant device is in a stabilized position. The method where: the one or more staple comprises a first staple and a second staple; the first staple having a first staple head positioned near a distal end of the cage; and the second staple having a second staple head positioned near a proximal end of the cage; the first staple head and the second staple head are each positioned within an outer wall of the cage; and the first staple head and the second staple head are configured to engage both the first bone portion and the second bone portion of the vertebral body when the implant device is in a stabilized position. The method where: the one or more staple comprises a first staple and a second staple; the cage comprises a first cage section coupled to the first staple and a second cage section coupled to the second staple; the first staple having a first staple head positioned near a distal end of the cage where the first staple head engages both the first bone portion and the second bone portion when the implant device is in a stabilized position; and the second staple having a second staple head positioned near a proximal end of the cage where the second staple head engages both the first bone portion and the second bone portion when the implant device is in a stabilized position. intravertebral applications.

Intravertebral use of the disclosed implant system is intended to restore foraminal height and treat vertebral body wedging, which result from microfractures and collapse of the vertebral body endplates. These microfractures occur because the collapsed disc creates abnormal stress areas in the vertebral body. The resultant vertebral body wedging, secondary to the microfractures, creates both sagittal and coronal deformity, causing back pain thru misaligned facet joints and leg pain due to foraminal stenosis. The source of the back pain can be confirmed by injecting diagnostic local anesthetic agents around the painful facet joint. Correction of these deformities in the vertebral body via osteotomy and placement of the vertebral implant will reduce the back and leg pain by realigning the facet joints and opening the foramen in this select group of patients. This is analogous to the use of high tibial osteotomies for treatment of knee arthritis. The implant design allows for careful and patient-specific sagittal and coronal alignment correction to prevent the clinical outcomes of misalignment.

This osteotomy procedure and implant device can relieve pain symptoms while maintaining lumbar spine mobility and prevent or delay adjacent level disease. The implant device does not have any motion itself but reestablished proper spinal alignment while preserving the intervertebral disc above and below the operated level.

With the disclosed implant system, a vertebral body osteotomy stabilized with the implant device can correct the wedged segment of the spine through the vertebral body. This opens the foramen and relieves the pinched nerve and therefore relieves the patient's radiculopathy symptoms. The implant design allows for careful and patient-specific sagittal and coronal correction to prevent the clinical outcomes of spinal misalignment.

This technology will bridge the gap between a minimally invasive decompression without fusion and more extensive decompressions requiring a fusion procedure and lead to an improved quality of life when compared to current standard surgical techniques and technology. The patient will have relief from back and/or leg pain without a loss of spine mobility, which can significantly reduce or eliminate the risk of adjacent level accelerated degeneration in the other levels of the spine. The custom alignment created with the implant device can prevent the clinical outcomes of spinal misalignment.

Examples of the implant system may comprise a vertebral implant device configured to alter a distance between a superior endplate surface plane and an inferior endplate surface plane of a vertebral body.

### Intervertebral Applications

Intervertebral use of the disclosed implant system is intended to fuse opposing vertebral bodies to eliminate painful motion and/or to restore anatomic alignment, height and stability to the spine following spinal decompression. This fusion eliminates motion between vertebrae and also prevents the irritation and stretching of nerves and surrounding ligaments and muscles.

Intervertebral use of the implant system generally provides an implant that is able to be secured to the inferior and superior endplates of two opposing vertebrae to facilitate a fusion. Dimensions of components of the implant system may also be shaped to provide patient-specific sagittal and coronal alignment to prevent the clinical outcomes of misalignment.

In some examples, the implant system comprises an intervertebral implant device configured to join one vertebral body to another vertebral body.

### Applications with Other Joints

Implant devices similar in design to the above implant systems may be used as an arthrodesis implant device in an arthrodesis procedure for other joints. As done for the joining of two vertebrae, an implant device may be provided that is configured to be secured to opposing sides of adjoining bones in a joint to fuse those bones. Stabilization may also be used to correct alignment of the bones of the joint.

In some examples of the implant system, the implant system comprises an arthrodesis implant device configured to join one bone to another bone.

Other objects, features, and advantages of the systems and techniques disclosed in this specification will become more apparent from the following detailed description of embodiments in conjunction with the accompanying drawings.

### BRIEF DESCRIPTION OF THE SEVERAL VIEWS OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A shows the sagittal, coronal, and transverse planes of the human body;

FIG. 1B illustrates the different axis and placement approach trajectories used with an example of an implant systems;

FIGS. 2A-2D show an example embodiment of an implant device;

FIGS. 3A and 3B show an example embodiment of the implant device as configured to be manipulated by implant system tools;

FIGS. 4A and 4B show an example posterior lateral approach trajectory for positioning the implant device;

FIGS. 5A-5C show an example posterior later approach trajectory for positioning the implant device;

FIGS. 6A-6C show top views of the positioning of the implant device;

FIGS. 7A-7D show top views of the positioning of the implant device with a puller assembly;

FIGS. 8A-8F show top and perspective views of an example embodiment of the implant device;

FIGS. 9A-9F show different views of the example embodiment of the implant device showing different positions of the staples;

FIG. 10 shows a view of the implant device secured to a vertebral body;

FIGS. 11A-11D show another example embodiment of an implant device;

FIGS. 12A-12F show top and perspective views of this example embodiment of the implant device;

FIGS. 13A-13C show different views of this example embodiment secured in the vertebral body;

FIGS. 14A-14B show an example embodiment of an insertion handle assembly alone and positioned with a vertebral body;

FIGS. 15A-15C shows an example embodiment of the insertion handle assembly being used to position an implant device in the vertebral body;

FIGS. 16A-14B show different views of another example embodiment of an implant device;

FIGS. 16C-16G show details of the implant device and the coupling mechanism;

FIGS. 17A and 17B show details of an example keyway mechanism;

FIGS. 18A-18D show different views of another example embodiment of an implant device and details of the coupling mechanism;

FIGS. 19A-19D show different views of an example embodiment of a bi-lateral implant device;

FIGS. 20A-20M show additional details of implanting and actuating the bi-lateral implant device;

FIGS. 21A and 21B show different views of this example embodiment secured in the vertebral body;

FIGS. 22A-22C show another example embodiment of an implant device:

FIGS. 23A-23D show different views of this example embodiment as positioned on a vertebral body portion;

FIGS. 24A-24I show details of an example embodiment of positioning and actuating this implant device;

FIGS. 25A and 25B show different views of another example embodiment of an implant device together with an insertion handle assembly;

FIGS. 26A-26D show different views of one cage section of this example embodiment;

FIGS. 27A and 27B show additional details of the coupling mechanism and actuating this implant device;

FIGS. 28A and 28B show additional details of actuating this implant device;

FIGS. 29A-29C show views of this example embodiment of the implant device;

FIGS. 30A and 30B show examples of positioning this example embodiment of the implant device with an insertion handle assembly;

FIGS. 31A-31D show additional views of positioning this example embodiment in the implant device; and

FIGS. 32A-32F show different views of this example embodiment secured in the vertebral body.

### DETAILED DESCRIPTION OF THE INVENTION

COPYRIGHT NOTICE: A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. The following notice applies: Copyright © 2020-2025, Foundation Surgical Group, Inc., All Rights Reserved.

Implant systems and methods of use will now be described in detail with reference to the accompanying drawings. Notwithstanding the specific examples set forth below, all such variations and modifications that would be envisioned by one of ordinary skills in the art are intended to fall within the scope of this disclosure. The implant systems and methods may be used as orthopedic implant systems such as, but not limited to, an intravertebral implant system for use in intravertebral applications, an intervertebral implant system for use in intervertebral applications and an implant system for arthrodesis procedures for other joints throughout the body. The implant systems and methods may comprise an orthopedic implant device such as, but not limited to, an intravertebral implant device for intravertebral applications, an intervertebral implant device for intervertebral applications or an implant device for arthrodesis procedures for other joints throughout the body.

Foraminal narrowing is a specific type of spinal stenosis, a spinal condition that occurs when the open spaces between the vertebra (the foramina) narrow. The foramina are bony passageways located between the vertebrae on either side of the spine. Their primary purpose is to provide an exit path for nerves leaving the spinal cord and traveling to other parts of the body.

Minimally invasive spine (MIS) surgery without fusion is generally intended to relieve pressure being applied to the spinal nerves—often a result of conditions such as spinal instability, bone spurs, herniated discs, scoliosis, or spinal tumors. In cases where extensive decompressions are required to accomplish the goal of relieving pain, a fusion may become necessary.

Fusion of opposing bones of a joint result in a permanent connection of the bones of the joint to eliminate motion between them. All fusions, including spinal fusion, involve techniques designed to mimic the normal healing process of broken bones where an implant device may be used to hold the vertebrae together, so they can heal into one solid and immobile unit.

Embodiments of the disclosed implant systems may be configured to correct vertebral body deformity in the coronal, sagittal, and axial plane (if needed).

The system may be suitable for indirect foraminal decompressions that require more than a MIS procedure but less than a large decompression and fusion. Embodiments of the disclosed implant systems may be used for 1 and 2 vertebral body interventions.

In some embodiments, the implant device generally acts as an opening wedge osteotomy spacer and uses the shape of implant components, such as cage surface planes, to alter the alignment of the vertebral body of a mammalian body.

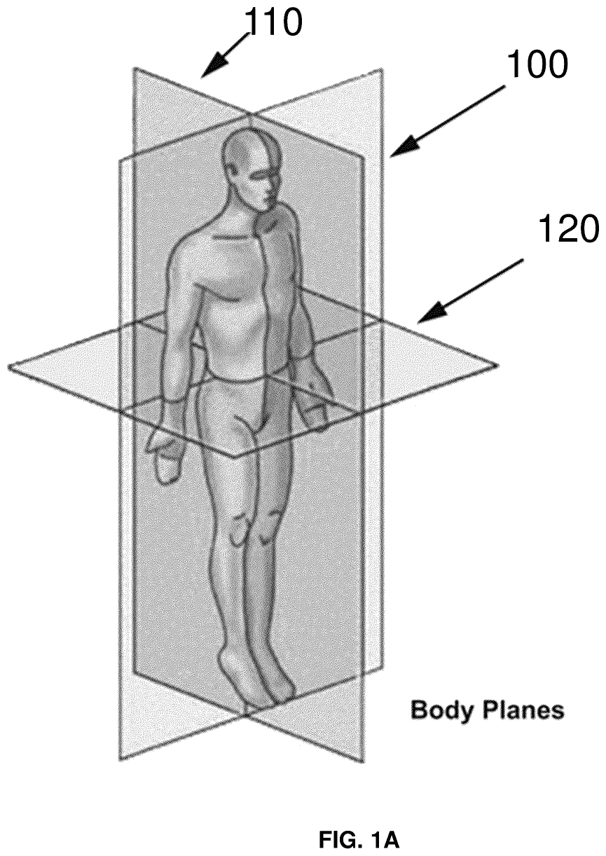

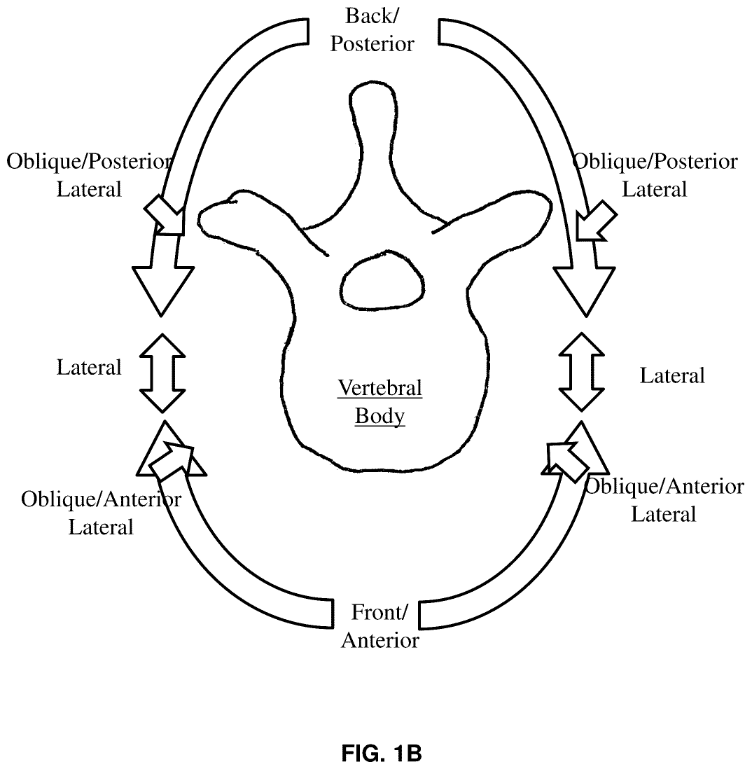

Referring to FIG. 1A showing the sagittal 110, coronal 100 and transverse 120 planes of the human body, embodiments may be used to correct alignment of the spine in the sagittal (110) and coronal (100) planes.

Referring to FIG. 1B, the implant system may be inserted and positioned at and from different angles relative to the vertebral body. For example, these angles may be the approach trajectory angles used in PLIF, TLIF and posterior lateral surgical procedures. The placement and configuration of the implant components dictate the different alignment surface angles of the vertebral bodies. In this description, placement trajectories of back and posterior generally mean the range of trajectories from one lateral position of the vertebral body to the other and front and anterior generally means the range from one lateral position of the vertebral body to the other. As shown, within these back/posterior trajectories, some embodiments of the implant system may be implanted through lateral, posterior, posterior lateral, or posterior oblique approach trajectories.

In some embodiments, the implant system is configured for use in posterior and/or posterior lateral procedures for implanting implant devices at spine locations T1 to S1.

In some embodiments, the implant system is configured for use in posterior and/or posterior lateral procedures for implanting implant devices in a spine levels from T11 to S1 using the Wiltse approach trajectory.

In some embodiments, the implant system may have components customized to be sized for specific patients and uses. For example, the implant system may have a cage that is custom sized from patient data to fit that specific patient.

In some embodiments, the implant system is configured to preserve the spinal vascular system.

In some embodiments, the configuration of the implant system allows for stabilization and bone fusion within the vertebral body after placement.

In some embodiments, the implant system is configurable. For example, the implant system may be configured to provide different alignments to vertebral bodies and the spine. For example, the implant system may provide configurable dimensions such as different height and angles of the cage surfaces to provide different cage surface planes and different sagittal and coronal angular correction when positioned in the vertebral body.

In some embodiments, the implant system may be a modular system including a self-stabilizing cage which includes deployable securing elements.

In some embodiments, the implant system may be pre-packed with bone graft (autogenous, allogenic, or synthetic) and the implant system may be configured to allow additional graft material to be post-packed, injected or otherwise placed after positioning of the implant within the vertebral body.

In some embodiments, provisions may be made to couple the implant system to other constructs such as rod/cord-screw systems, flexible tethers, and plate systems.

In some embodiments, the implant system generally comprises a cage with a staple and a second staple. The staple and the second staple may be on opposing sides of the cage to secure the cage to bone. In some embodiments the staple and second staple may have features and a coupling mechanism that allows the staples to be inserted, extended, deployed, and stabilized or secured to the bone.

In some embodiments, the staples of the implant system may have extension, deployment and retracting features that allow the staple to be moved through multiple positions to secure the implant device to the bone. The movement features may allow the staples to be easily moved between an insertion position, an extended position, a deployed position, and a stabilized position. For both staples, these different positions of the staple describe both the rotational alignment of the staple head and the location of the staple head relative to other elements of the implant device.
Insertion position: In the insertion position, alignment of the length of the staple head is in a neutral alignment, generally the orientation of the length of the staple head being coplanar or parallel to a plane extending along the transverse axis of the cage. In this position, the longitudinal location of the staple head relative to the longitudinal axis of the cage is the location as the implant device is being positioned for implanting. In some embodiments, the longitudinal location of the staple head is generally positioned in a non-extended/insertion location close to the distal end of the cage. In some embodiments, the insertion location of the staple head position may have the staple head in an extended location extended away from the cage. In some embodiments, portions of the staple head may be received in a protective recess on the cage.
Extended position: In the extended position, the alignment of the staple head is in the neutral alignment, generally parallel to the transverse axis of the cage. In this position, for embodiments with staples outside of the cage, the longitudinal location of the staple head relative to the cage longitudinal axis is in an extended location extended away from the cage. For embodiments with staples outside of the cage, the extended position generally extends the location of the distal staple head from the cage longitudinally so that the staple head and the staple tines extend beyond the sidewalls of the bone.
Deployed position: In the deployed position, the staple head is rotated to a non-neutral alignment that is other than parallel to the transverse axis of the cage. In this position, the non-neutral alignment of the staple head may be at any angle relative to the insertion and extended position sufficient to allow the staple tines of the staple to be positioned to engage the sidewalls of the bone. The longitudinal location of the staple head relative to the longitudinal axis of the cage is in: (1) for embodiments with staples outside of the cage, an extended location extended away from the cage sufficient to allow the staple tines of the staple head to extend beyond the bone, or (2) for embodiments with staples within the cage, generally at the longitudinal location of the insertion position. Preferably, the non-neutral alignment in the deployed position is about 90 degrees from the neutral alignment to maximize engagement with the bone.
Stabilized position: In the stabilized position, alignment of the length of the staple head is not parallel to the transverse axis of the cage and sufficient to allow the staple tines of the staple to engage the walls of the bone. In this position, the alignment of the staple head is generally in the non-neutral alignment of the deployed position. In this position, the longitudinal location of the staple head relative to the cage is in: (1) for embodiments with staples outside of the cage, a retracted location retracted towards the cage sufficient to allow the staple tines of the staple head to engage or embed themselves in the bone to mechanically engage and stabilize the staple head and the cage to the bone, or (2) for embodiments with extendable staples within the cage, an extended location away from the center of the cage sufficient to allow the staple tines of the staple head to further engage or embed themselves in the bone to mechanically engage and stabilize the staple head and the cage to the bone, or (3) for embodiments with non-extendable staples within the cage, generally at the longitudinal location of the deployed position sufficient to allow the staple tines of the staple head to engage or embed themselves in the bone to mechanically engage and stabilize the staple head and the cage to the bone. In this position, the second staple head may provide a counter force to the first staple.

To support the above positions, the staples may comprise the staple head and a staple shaft. The staple shaft may be configured to move and rotate the staple head through the above positions relative to a longitudinal axis of the staple. The staple shaft may rotate about the staple longitudinal axis and the staple shaft may translate along the staple longitudinal axis. For example, the staple shaft may be rigidly coupled to the staple head and configured to move the staple head from an extended position to a deployed position by a rotation of the shaft and the staple head. As another example, the staple shaft may be configured to move the staple head from an insertion position to an extended position by slidably moving the staple shaft through a bore of the cage and extending the staple head away from the cage. As another example, the staple shaft may be configured to move the staple head from an insertion position to an extended or other position by a combination of sliding and rotating the staple shaft. The staple shaft may also be configured to move the staple head from a deployed position to a stabilization position by slidably moving the staple shaft through a bore of the cage and retracting the staple shaft and staple head towards the cage.

To support the positioning and movement of the staple shaft and staple head through different alignments and longitudinal locations, a coupling mechanism may be provided. The coupling mechanism generally includes the implant system components that may be manipulated to move, of any combinations of features of implant system components that may influence the movement of, implant system components relative to each other. For example, the coupling mechanism may be implant system components configured to be engaged by one or more tool and the coupling mechanism moves the staple shaft and staple head through the different alignment and longitudinal locations described herein. The alignments are generally the rotational positions of the staple head and the longitudinal locations are generally the locations of the staple head as it translates relative to the cage.

The coupling mechanism may be configured to move the staple through the different longitudinal locations. For example, to support the movement of the staple head through the different longitudinal locations, the staple shaft may also be configured to translate to move the staple head from an insertion to an extended longitudinal location by having the staple shaft mate with a coupling device to extend the staple head away from the cage. In this example, the staple shaft may have an externally threaded coupling portion that mates with a coupling element like an internally threaded nut and a rotation of the nut engages the threads to move the staple shaft.

In some embodiments, the coupling mechanism may also be configured to move the staple through the different alignments. For example, the coupling mechanism may also include a keyway mechanism to provide features that allow the coupling mechanism to allow and restrain movement of the staple shaft when the staple shaft is in different positions. The keyway mechanism may be any combination of elements or any combination of features of elements that can restrain the alignment or longitudinal locations of the staple in one position and can allow for alignment or longitudinal location changes in another position. For example, the keyway mechanism may allow the staple shaft to translate along its longitudinal locations without changing its alignment when the staple shaft is in one position and the keyway mechanism may also allow the staple shaft to move into another alignment without translation when the staple shaft is in another position.

In some embodiments, the coupling mechanism may also be configured to influence the movement of other implant device components. For example, the coupling mechanism may include an engagement mechanism to influence the movement of additional implant device components. The engagement mechanism may be any combination of elements or any combination of features of elements that allow one component of the coupling mechanism to move another implant device component. For example, the staple shaft may have an engagement portion that mates with an engagement portion on a second staple to allow the second staple to alter its alignment.

To support the positioning and movement of the staple head through the different alignments and longitudinal locations of the above positions, the coupling mechanism may also be configured to move the staple head from an insertion to an extended position by having a threaded staple shaft and rotating the coupling element with mating threads to extend the staple head away from the cage. The coupling mechanism may also be configured to move the staple head from the extended position to a deployed position by configuring the staple shaft to rotate the staple shaft and the staple head into the deployed position. The coupling mechanism may also be configured to move the staple head from the deployed position to a stabilization position by having a threaded staple shaft and rotating the coupling element with mating threads to retract the staple head towards the cage. The coupling mechanism may also be configured to move the staple head from the deployed position to a stabilization position by having a threaded staple shaft engage mating threads of the coupling element and rotating the coupling element to retract the staple head towards the cage.

To support the securing of the staple head to the bone when implanted, the staple head the staple may have a first staple portion and a second staple portion whereby when the staple head is in the deployed position, the first staple portion is configured to extend above the cage and engage a first bone positioned above the cage and the second staple portion is configured to extend below the cage and engage a second bone positioned above the cage.

In some embodiments, the staple is positioned on the distal side of the implant to be secured to the distal sidewall of the vertebral body yet control of the positioning of the staple is done by manipulating system elements and features accessible on the proximal side of the implant. These features are particularly beneficial for vertebral procedures where the implants are inserted and secured from a lateral or an oblique approach angle trajectory and the implant is implanted across the vertebral body and secured to both lateral sidewalls of the vertebra. These procedures include the ATP approach trajectory to access the vertebral body and implant the implant device.

In some embodiments, components of the implant device may be 3D printed as one unit.

In some embodiments, components of the implant device may include lattice or other surface configurations to encourage bone growth and secure the implant device to bone. For example, the cage may be made with portions having lattice structures or it may have a percentage lattice volume such as about 40-70 percent.

The implant device may be manufactured from any suitable material including commercially pure titanium, titanium alloy, polyetheretherketone or any other appropriate material, even allogenic bone. In one example, all of the components of the implant device are made of a surgical grade metal such as Titanium (e.g., ASTM F136 Wrought 6Al4V Ti for Implant). The implant device components may be manufactured utilizing conventional machining technology (e.g., milling and turning, mass media and/or electropolish finishing, color anodizing and passivation) or one of the several available methods additive manufacturing methods.

### Example Embodiments of the Implant System

It is understood that the disclosed implant systems and methods of use may be used with different orthopedic procedures. For illustration purposes only, and not for limitation, an example of the implant system used for intravertebral applications will be described and referred to as a vertebral implant system, an intravertebral implant system, a vertebral implant device and an intravertebral implant device. It is understood that implant systems consistent with this example may be used as intervertebral implant systems, intravertebral implant systems and implant systems configured for use in arthrodesis procedures. In this illustrative example, the implant system comprises a vertebral implant device configured for use as an intravertebral implant device. And in these examples, the positioning of the vertebral implant device will be made using approach trajectories such as those used in posterior, posterior lateral, PLIF, or TLIF procedures.

### External Staple Heads

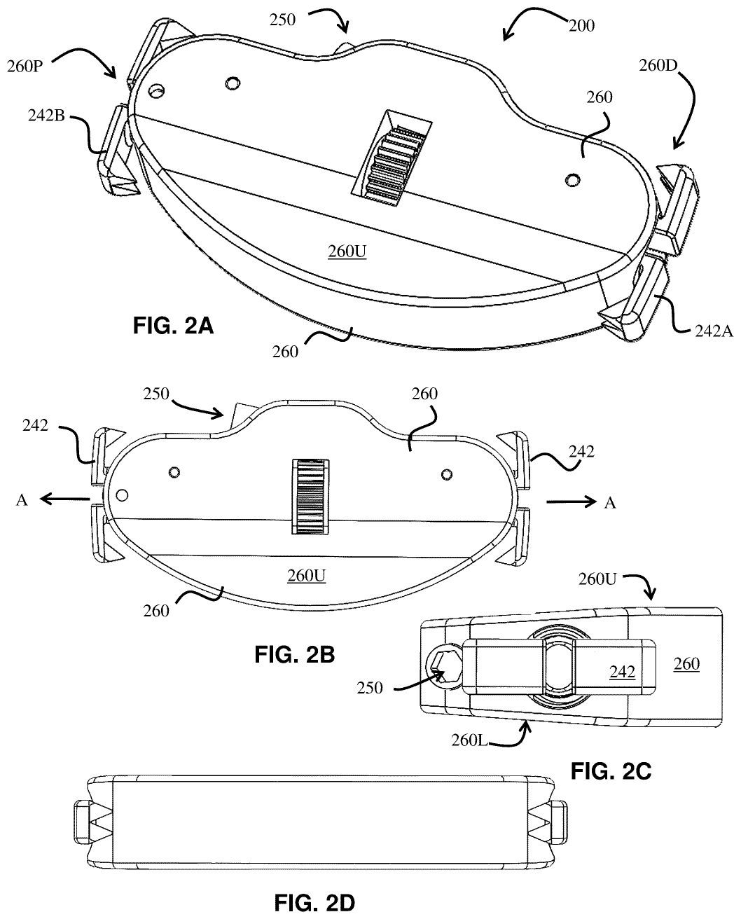

For illustration purposes and not for limitation, one example of the vertebral implant device is shown in FIGS. 2A-2D. FIGS. 2A-2D show an example embodiment of an implant device. In this example embodiment, the implant device has two external staples, external to the cage when in an extended, deployed, and stabilized position. FIG. 2A shows a top perspective view of the implant device, FIG. 2B shows a top view, FIG. 2C shows a side view and FIG. 2D shows a font view.

As shown in the example of FIGS. 2A-2D, the vertebral implant system generally comprises an intravertebral implant device 200 comprising a cage 260, one or more securing element configured to secure the cage to the vertebral body and a coupling mechanism to manipulate the securing element. The securing element may be any suitable element or combination of elements to secure the cage 260 and the implant device 200 to a superior and inferior portion of the vertebral body. In the example shown, the securing element may comprise one or more staple received in, and rotationally and longitudinally adjustable relative to the cage 260.

Referring to FIG. 2A, the securing element comprises one or more staple having a staple head 242A positioned outside and proximal to a distal end 260D of the cage 260 and staple head 242B positioned outside and proximal to a proximal end 260P of the cage 260. The cage 260 generally functions as a spacer between bones or between portions of a bone when implanted. The coupling mechanism 250 provides adjusting device features for the implant system to manipulate the one or mor staple and staple heads 242A and 242B relative to the cage 260. This extension and retraction caused by the coupling mechanism adjusts the locational relationship between the staple heads 242A and 242B and the cage 260 and other implant device elements.

It is understood that in some embodiments, the securing element may comprise a single staple and staple head with an opposing force to secure the cage 260 to the bone. The opposing force may be provided by an opposing plate or features of the cage that are able to provide an opposing force to the staple head.

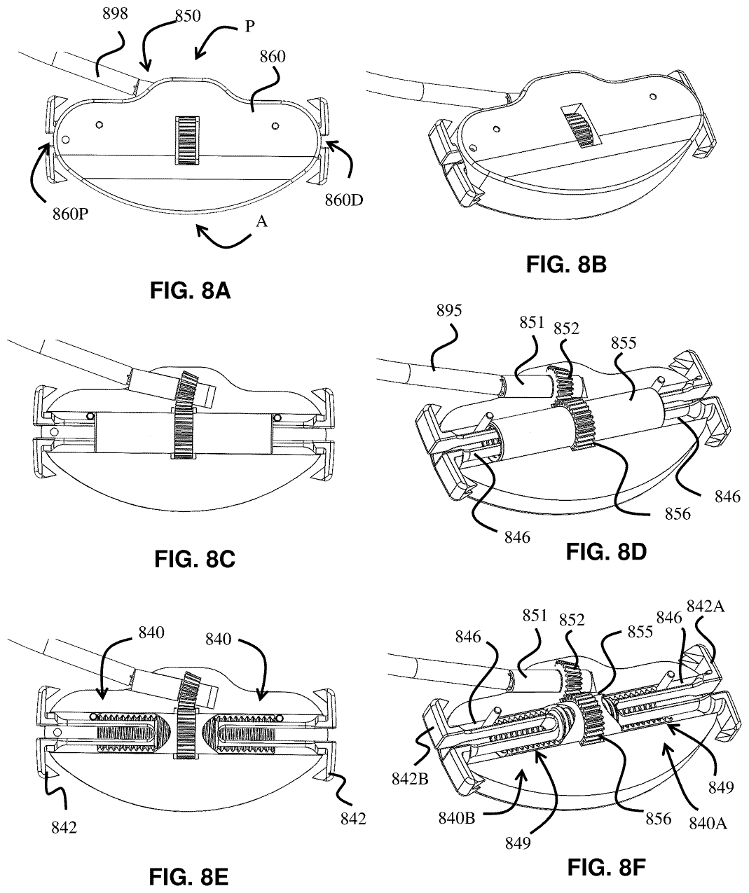

The staple generally comprises a staple head 242A having staple tines, a staple shaft, a coupling portion and an engagement portion. The staple head 242 may be part of a staple including a staple shaft (see FIGS. 8A-8F). The staple head 242A generally extends from a portion of the cage 260 to mechanically engage a sidewall of the bone to secure it and the cage 260 to the bone. It is understood that although the example of the staple head 242A and 242B illustrate a unitary staple head from one end to the other, in some embodiments, the staple head may be comprised of multiple components such as components hinged or otherwise coupled to each other. As shown, an opposing staple head 242B also provides features to secure itself and the cage 260 to the bone. Together, staple head 242A and 242B provide a compressive force to secure the cage 260 to the bone. The staple tines may be any shape to secure the staple in the vertebral body and they are generally stiff and if intended for intraosseous deployment, sharpened to cut through vertebral bone, if intended for external deployment, they will comprise smooth rounded surfaces with the exception of the bone facing staple tines. The coupling portion and the engagement portion generally interface with the coupling mechanism and provide a means to engage and to manipulate the staple. The coupling portion generally provides a means to extend and retract the staple head and the engagement portion generally provides a means to rotate the staple shaft and the staple head. In some embodiments, the coupling portion may provide the means to both translate and rotate the staple head.

The cage comprises a body generally having an upper surface 260U defining the cage upper surface plane for the implant device and the lower surface 260L defines the cage lower surface plane. The upper and lower surface planes, the space between them and the angles between them define the correction that can be provided through the implanting of the cage.

The coupling mechanism 250 is generally configured to mate with the cage and the staple shaft to move the staple shaft and the staple head through different longitudinal locations and alignments relative to the cage. In this example, the coupling mechanism is also configured to be engaged by engagement tools to engage and move the staple shaft and staple head through different rotational alignments.

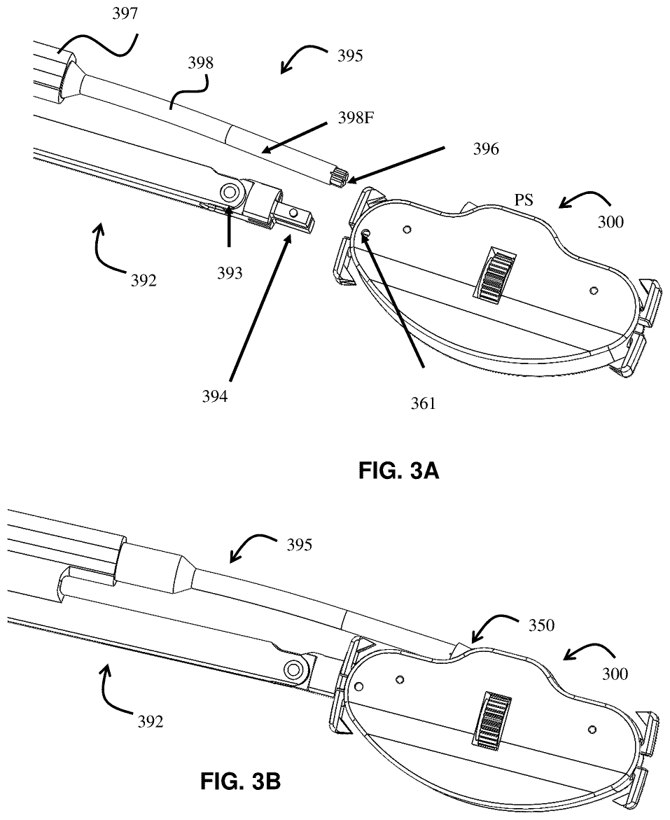

FIGS. 3A and 3B show an example embodiment of the implant device 300 as configured to be manipulated by implant system tools. FIG. 3A shows the implant device 300, an insertion handle assembly 392, and a staple drive handle assembly 395. The insertion handle assembly 392 is used for positioning and applying insertion force to implant device 300. The insertion handle assembly 392 may have tip 394 to removably couple with the implant device 300. For example, the tip 394 may be an expandable tip inserter with bosses to lock the tip of the insertion handle assembly into a mating implant recess. As shown the expandable tip inserter may have flexible forked tips with externally facing bosses that mate with recesses 361 at one of the ends of the cage. The insertion handle assembly 392 may be flexible to allow movement of the tip inserter when mated with the implant device. For example, the insertion handle may have a hinge 393 as shown. The staple drive handle assembly 395 is used to extend, rotate and retract staples. The staple drive handle assembly 395 may have a drive tool, such as a drive rod 398 that may have a flexible or jointed shaft section 398F and a handle 397 to move the drive rod 398. The drive rod 398 may also have a tip 396, such as an expandable tip, to mate and become coupled with the coupling mechanism. FIG. 3B shows how the insertion handle assembly is coupled to the proximal end of the implant device and the and the staple drive assembly is coupled to the coupling mechanism 350. As shown, the staple drive handle assembly 395 couples with the coupling mechanism 350 positioned on the posterior PS side of the cage.

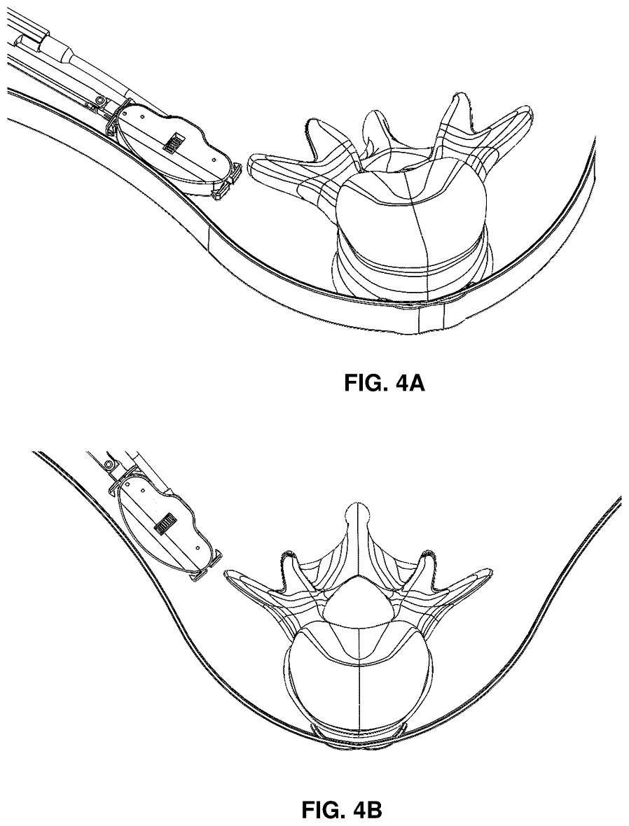

FIGS. 4A and 4B show an example posterior lateral approach trajectory for positioning the implant device with the insertion handle assembly and the staple drive handle assembly. Malleable retractors are shown as may be positioned during an implant procedure.

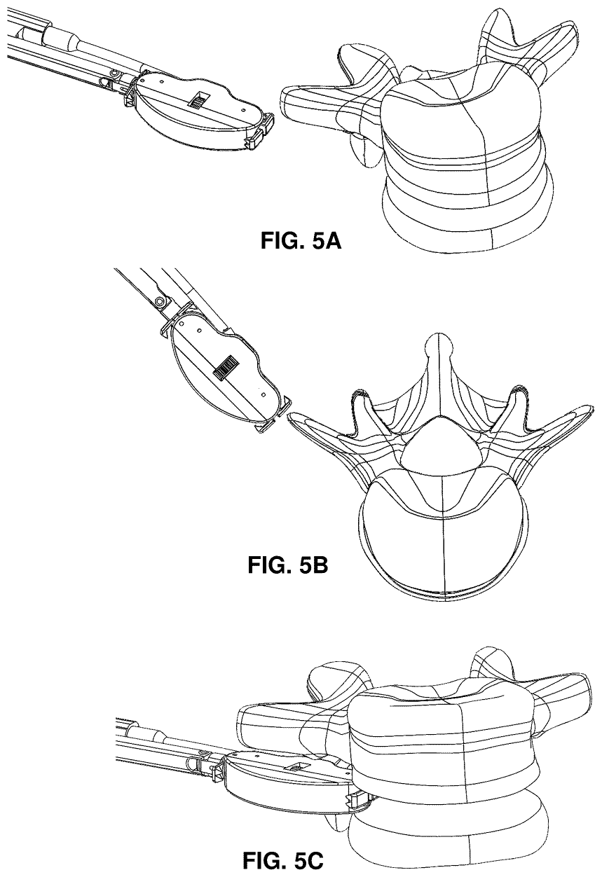

FIGS. 5A-5C show an example posterior lateral approach trajectory for positioning the implant device. The malleable retractors are removed for clarity. FIG. 5A shows a top perspective view of the approach trajectory, FIG. 5B shows a top view of the approach trajectory and FIG. 5C shows the implant as being inserted into an osteotomy space in an intravertebral procedure.

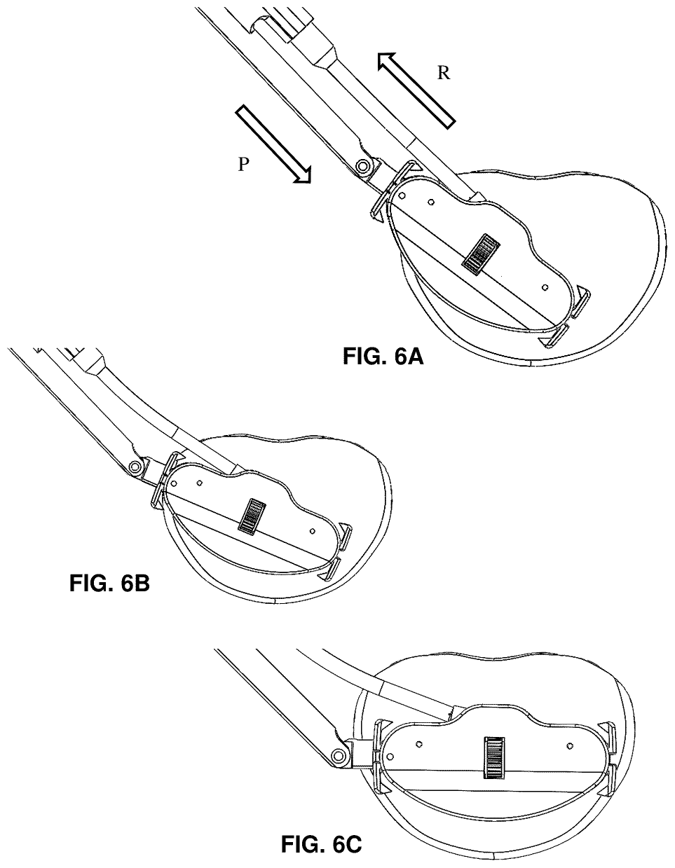

FIGS. 6A-6C show top views of the articulation and positioning of the implant device with the insertion handle assembly. FIG. 6A shows the action of articulating the implant device by pushing on the insertion handle assembly in direction P and retracting the driver of the staple drive handle assembly in direction R. The implant device is able to be articulated by the hinge of the insertion handle assembly and the flex of the staple drive handle assembly. FIGS. 6B and 6C show further articulation of the implant device. When the implant device is positioned, as in FIG. 6C, the insertion handle assembly may be removed from the implant device.

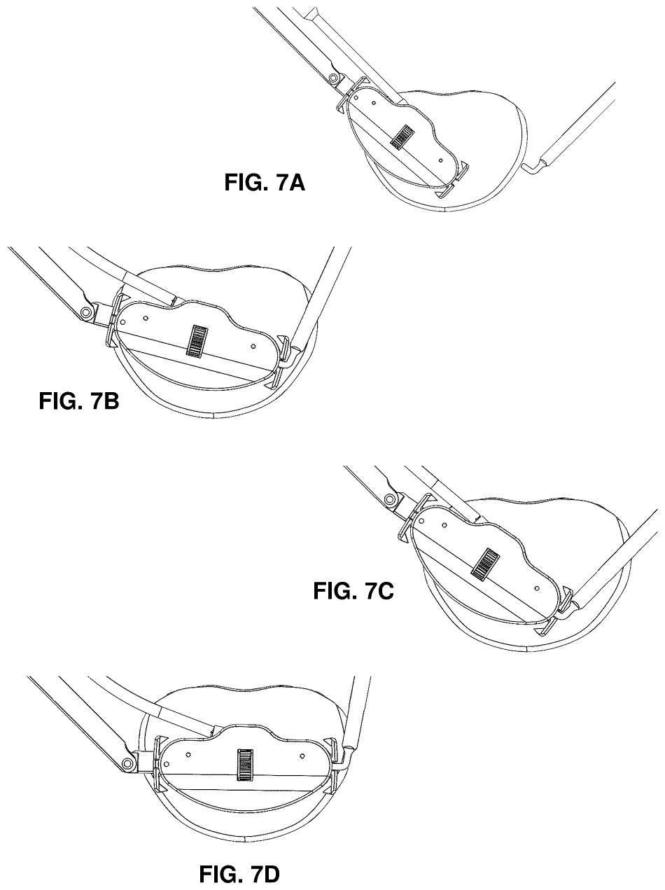

FIGS. 7A-7D show top views of the articulation and positioning of the implant device with insertion handle assembly aided by a puller assembly. The puller assembly is configured to aid positioning of the implant device. FIG. 7A shows the positioning of the implant device with the insertion handle assembly and the positioning of the puller assembly on the opposite side of the vertebra. FIG. 7B shows the action of articulating the implant device by pushing on the insertion handle assembly, retracting the driver of the staple drive handle assembly and further positioning with the puller assembly. The implant device is further articulated by the push and/or pull of the puller assembly on the distal side of the implant assembly. FIGS. 7C and 7D show further articulation of the implant device. When the implant device is positioned, as in FIG. 7D, the insertion handle assembly and the puller assembly may be removed from the implant device.

FIGS. 8A-8F show top and perspective views of an example embodiment of the implant device. FIG. 8A shows the cage 860 having a proximal end 860P, a distal end 860D, a posterior side P, and anterior side A, and a staple drive rod 898 coupled with the coupling mechanism 850 at the posterior side P of the cage 860. FIG. 8B shows a perspective view of the embodiment in FIG. 8A. FIG. 8D shows a perspective view of the embodiment in FIG. 8C, both with a portion of the body section opened to show the internal components of the implant device. FIG. 8F shows a perspective view of the embodiment in FIG. 8E, both with a portion of the body section and a portion of the drive cylinder opened to show internal components. As shown, the drive rod rotation axis and the drive coupler rotation axis are non-parallel and not aligned with the staple rotation axis or the staple translation axis.

As shown in FIGS. 8C and 8D, the coupling mechanism comprises a drive coupler 851 and a drive cylinder 855. The drive mechanism is configured to be engaged by the drive rod 898 which engages the drive coupler 851 which engages the drive cylinder 855 which in turn engages the staple shafts 846. In the embodiment shown, the drive coupler 851 is generally cylindrical shaped to receive the tip of the drive rod 898 whereby a rotation of the drive rod 898 rotates the drive coupler 851. As one means to engage the drive cylinder 855, the drive coupler 851 may have external teeth 852 around its outer circumference and these teeth are configured to engage external teeth 856 around the outer circumference of the drive cylinder. As an example means for the drive cylinder 855 to engage the staple shafts 846, as shown in FIGS. 8E-8F, the drive cylinder 855 may also be internally threaded so that the threads mate and engage external threads on a coupling portion 849 of the staple shafts 846. Operationally, by rotating the drive coupler 851 with the drive rod 898, the external teeth 852 of the drive coupler 851 engage the external teeth 856 of the drive cylinder 855 to rotate the drive cylinder 855. When the drive cylinder 855 rotates, the internal threads of the drive cylinder 855 rotate to engage the mating external threads of the coupling portion 849 staple shaft 846. The rotation of the staple shafts 846 are constrained during a portion of the translation so the staple shaft 846 and the staple head 842 is forced to translate. When the staple shaft 846 has translated to a translation stop, the staple head 842 is positioned in the extended position. At the translation stop, the staple shafts 846 no longer translate but are urged to rotate with continued rotation of the drive rod 898 and the drive cylinder 855. The staple shafts 846 rotate until they encounter a rotation stop. Generally, this rotation stop positions the staple heads 842 in the deployed position. The drive coupler 851 can then be rotated in the other direction, and with the staple shaft 846 configured to encounter a rotation stop, the staple shaft 846 translates in the other direction.

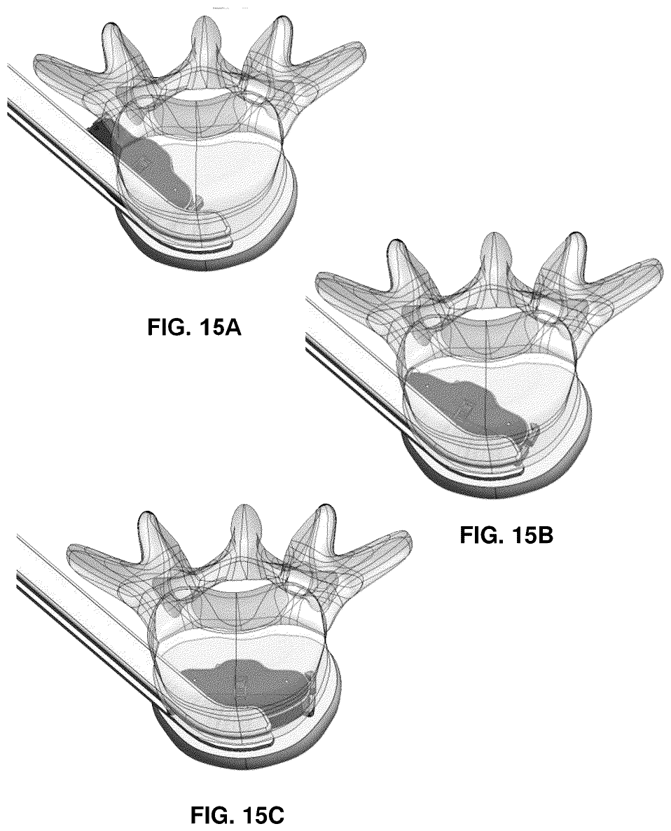

The means to constrain the translation and rotation of the staple shaft and to stop translation and rotation of the staple shaft may be provided by any suitable means. For example only, and not for limitation, one means to constrain and stop movement of the staple shaft comprises the keyway mechanism, an example of which is described herein and illustrated by FIG. 15, may be incorporated into the cage, the staples and the coupling mechanism of this embodiment.

In this embodiment, as shown, the drive coupler rotates along a rotation axis that is non-parallel to the rotation axis of the staple and the drive cylinder.

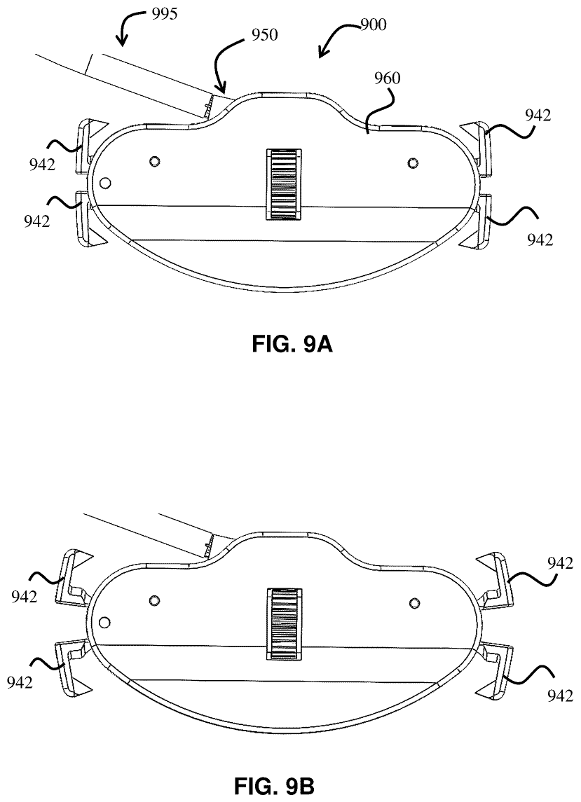

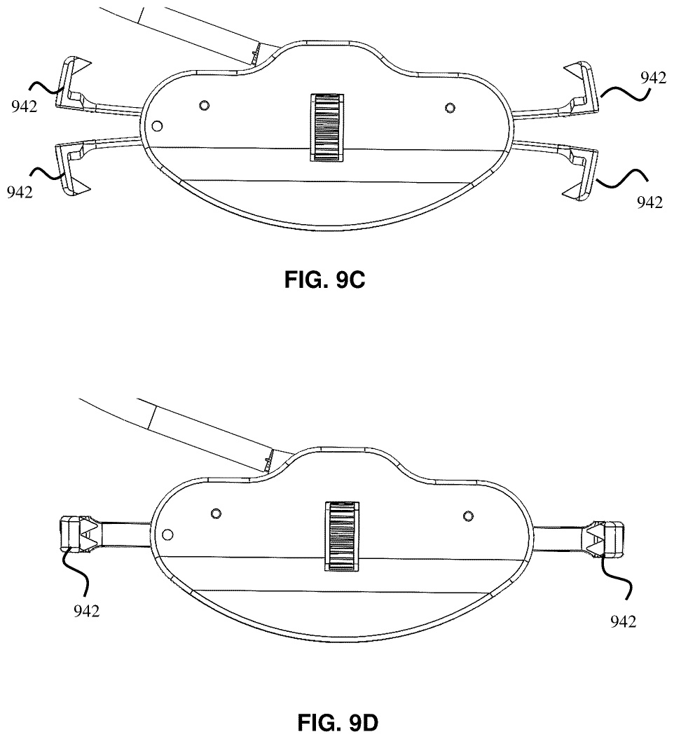

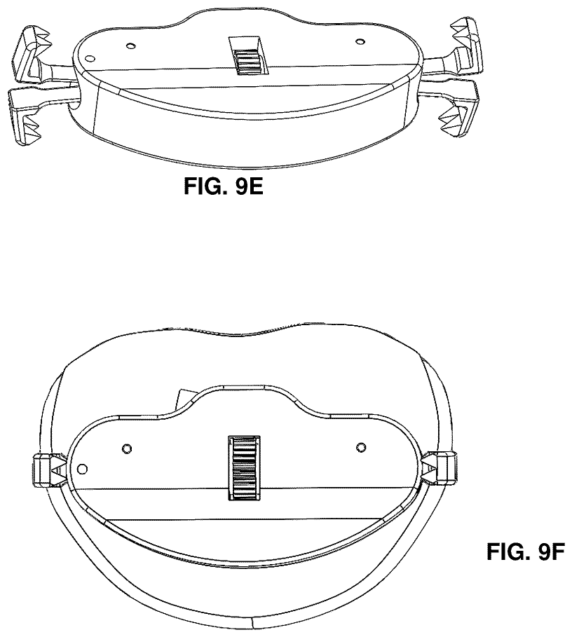

FIGS. 9A-9F show different views of the example embodiment of the implant device 900 showing different positions of the staple heads 942 showing deployment of the staple heads 942 with the vertebral body hidden for clarity. The positioning of the staple heads 942 is done by manipulating the staple drive handle assembly. FIG. 9A shows the coupling of the staple drive handle assembly 995 with the cage 960 with the coupling mechanism 950. The staple drive handle assembly 995 has a rotatable drive rod that, when rotated, engages with a coupling mechanism 950 that moves the staple heads 942 longitudinally and rotationally. FIG. 9B shows that by rotating the drive rod, the coupling mechanism extends the staple outside of the cage and translates the staple heads 942 away from the cage. As the staple heads extend, the forks of the staples are elastically biased so that they splay open as a portion of the staple is unconstrained by a channel in the cage. FIG. 9C shows that the staple heads 942 will extend until encountering a translation stop. FIGS. 9D and 9E show that while continuing to rotate the drive rod and the coupling mechanism 950 once the internal translation stop is hit, the staple heads 942 then rotate because they can no longer translate/extend until they hit a rotational stop. This rotational stop generally stops the rotation of the staple heads 942 in the deployed position. And once the staple heads 942 are positioned, the drive rod can be rotated the other way to retract the staple heads 942. FIG. 9F shows that once the staple heads 942 are secured to the bone, in a stabilized position, the drive rod can be removed. FIG. 9F shows a top view of the implant device in a secured position to a vertebral body.

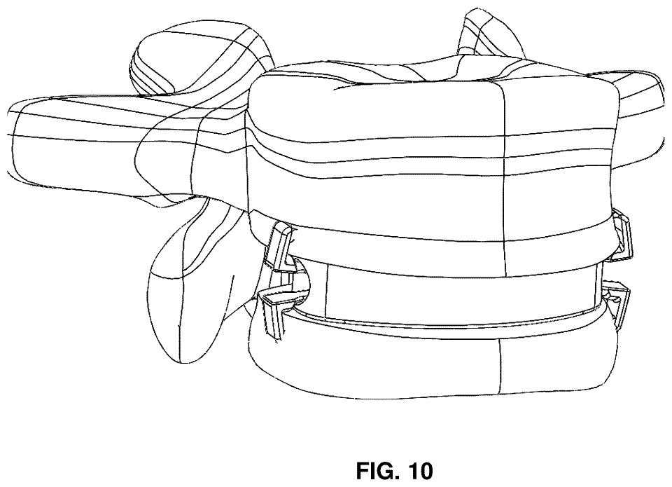

FIG. 10 shows a view of the implant device secured to a vertebral body.

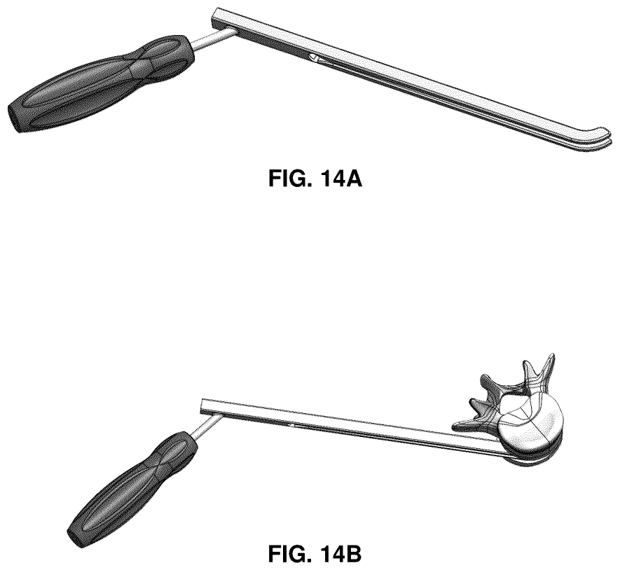

FIGS. 14A-14B show an example embodiment of an insertion handle assembly suitable for use with the implant device to insert and position the implant device in the vertebral body. FIGS. 15A-15C show the example embodiment of the insertion handle assembly with the implant device moving through the distal end of the channel of the insertion handle assembly and between the portions of the vertebral body.

Although the preceding description illustrates utilizing an implant device in an intrabody procedure, the same device and mechanisms may be used in an interbody procedure or an arthrodesis procedure.

### Internal Staple Heads

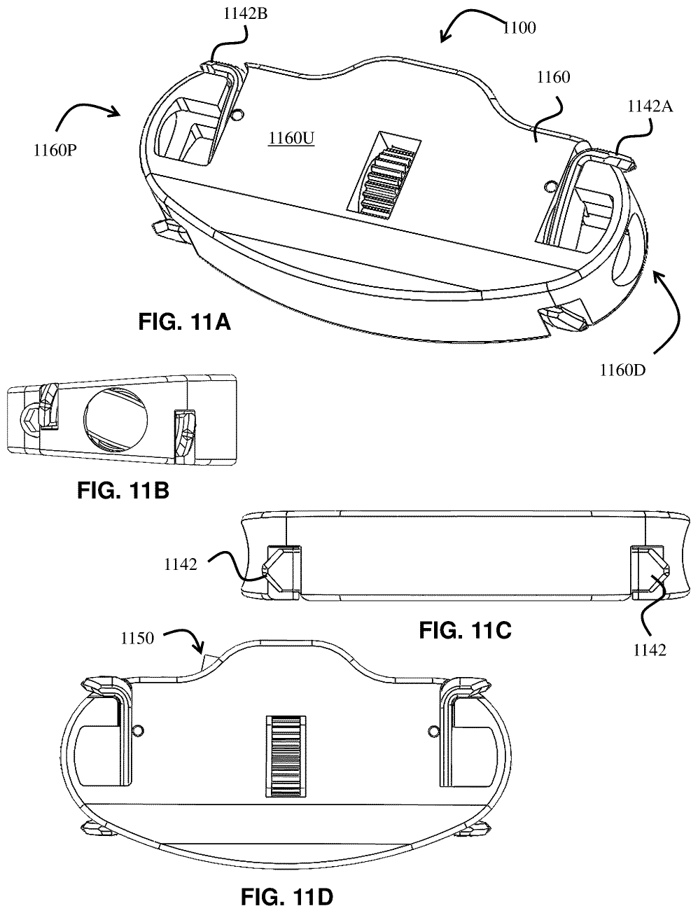

FIGS. 11A-11D show another example embodiment of an implant device 1100. In this embodiment, the staple heads 1142 are generally positioned near the distal end 1160D and proximal end 1160P of the cage 1160 but the staple heads 1142A and 1142B are within the outer walls of the implant cage and configured for intraosseous deployment. In this embodiment, the staple heads 1142A and 1142B and staple tines are stiff and sharpened to penetrate vertebral bone. The staple heads 1142A and 1142B may have tines that are positioned to extend from the staple heads 1142A and 1142B and outward from middle of the cage 1160 so they further engage and become embedded in the vertebral bone when they are extended. In this embodiment, the staple heads 1142A and 1142B can be rotated into the deployed position and extended away from the middle of the cage 1160 to further secure them in their stabilized position. FIG. 11A shows a top perspective view of this example embodiment, FIG. 11B shows a side view, FIG. 11C shows a front view, and FIG. 11D shows a top view. FIG. 11D shows the coupling mechanism 1150 on the posterior side of the cage. Consistent with the embodiments described herein and shown in FIGS. 2A-2D, the drive rod rotation axis and the drive coupler rotation axis are non-parallel and not aligned with the staple rotation axis or the staple translation axis.

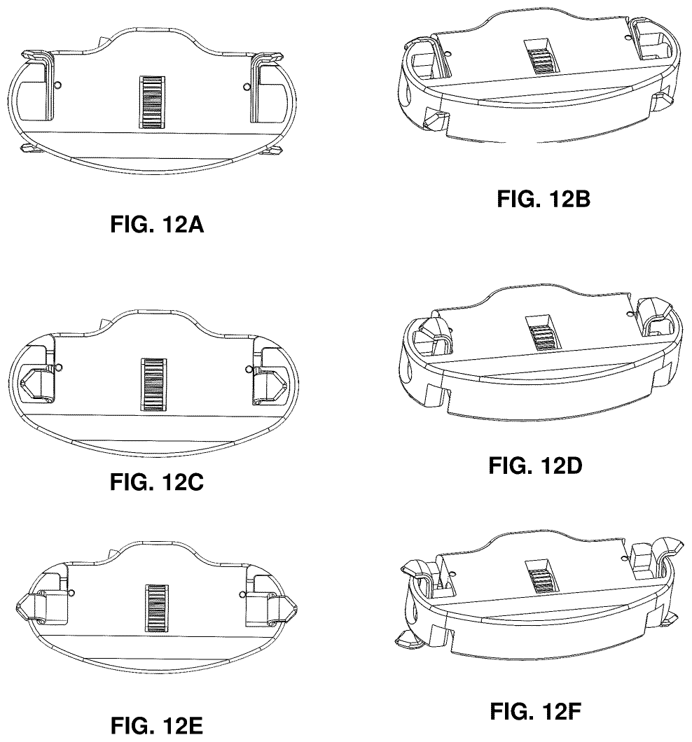

FIGS. 12A-12F show top and perspective views of this example embodiment of the implant device. FIG. 12B shows a perspective view of the embodiment in FIG. 12A. FIG. 12D shows a perspective view of the embodiment in FIG. 12C. FIG. 12F shows a perspective view of the embodiment in FIG. 12E.

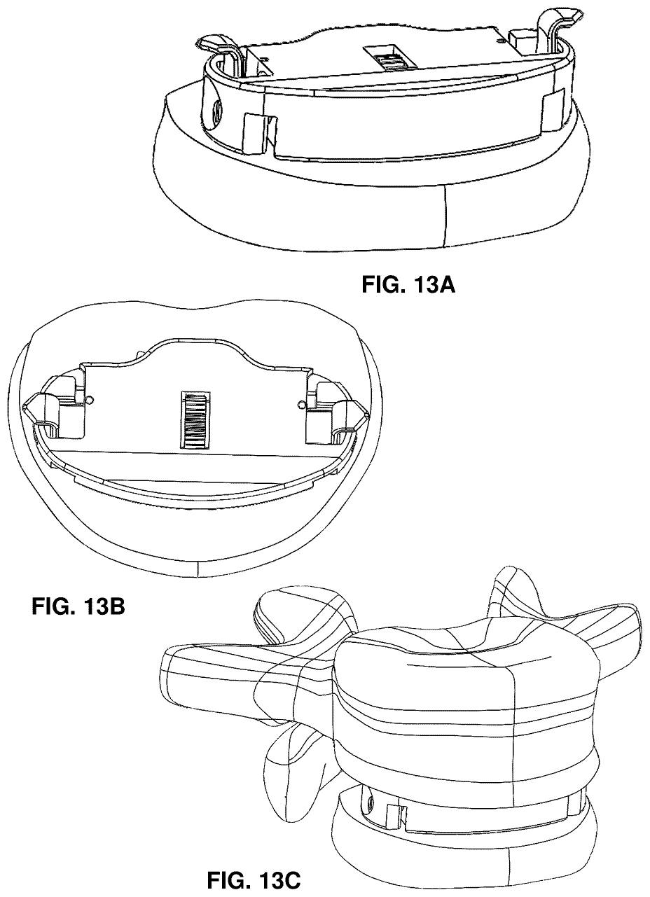

FIGS. 13A-13C show different views of this example embodiment secured in the vertebral body. FIG. 13A shows a perspective view of the implant device secured in the vertebral body without showing the top bone portion and FIG. 13B similarly shows a top view. FIG. 13C shows a perspective view of the implant device in a stabilized position within the vertebral body.

Operationally, this example embodiment manipulates the staples and staple head with coupling mechanism components similar to the embodiments of FIGS. 8A-8F. This embodiment moves the staple and staple heads by having a drive rod rotate which rotates the coupling mechanism, which engages and rotates the staples as described above for FIGS. 8A-8F. The drive coupler is configured to be engaged by the end of the drive rod. The drive coupler is engaged with a drive cylinder such that when the drive rod is rotated, the drive cylinder rotates and engages the staple shafts of the staples. In this example, the cylinder's internal threads engage the external treads of the staple shaft and friction causes the staple heads to rotate. When the staple head hits a rotation stop, such as a surface or edge of the implant device, the staple heads stop rotating generally in the deployed position. Further rotation of the drive rod overcomes the friction between the staple shaft and the drive cylinder and the staple heads translate and extend away from each other to the extended and stabilized position. The staple may also hit a translation stop such as an edge of the cage surface that stops the translation of the staple head.

This example embodiment with the staple heads positioned within the cage is different than the example embodiment with the external staple heads in that this embodiment initially rotates the staple head to hit a rotation stop and then the staples translate. The example embodiment described with external staple heads is constrained from rotating initially, hits a translation stop and then is configured to rotate into the deployed position.

In some embodiments, the distal and proximal walls of the cage may be configured to extend from the middle of the cage. This movement may be made by engagement with the coupling mechanism or other implant device components. This extension allows for portions of the outer walls of the cage to align with the edge of the cortical wall when the implant device is secured to the vertebra and provide structural support for the vertebra with the implant device.

This embodiment may be positioned in the vertebral body with tools such as the insertion handle assembly shown in FIGS. 14A-14B and FIGS. 15A-15C. As shown in FIG. 11D, the drive rod rotation axis and the drive coupler rotation axis are non-parallel and not aligned with the staple rotation axis or the staple translation axis.

And although the preceding description illustrates utilizing an implant device in an intrabody procedure, the same device and mechanisms may be used in an interbody procedure or an arthrodesis procedure.

### Rotating Staple Embodiment

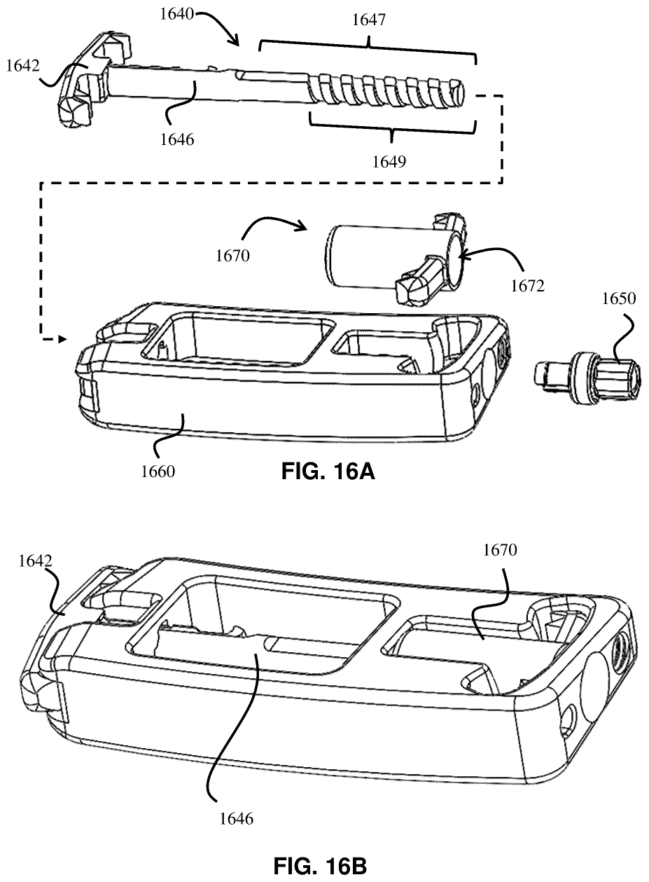

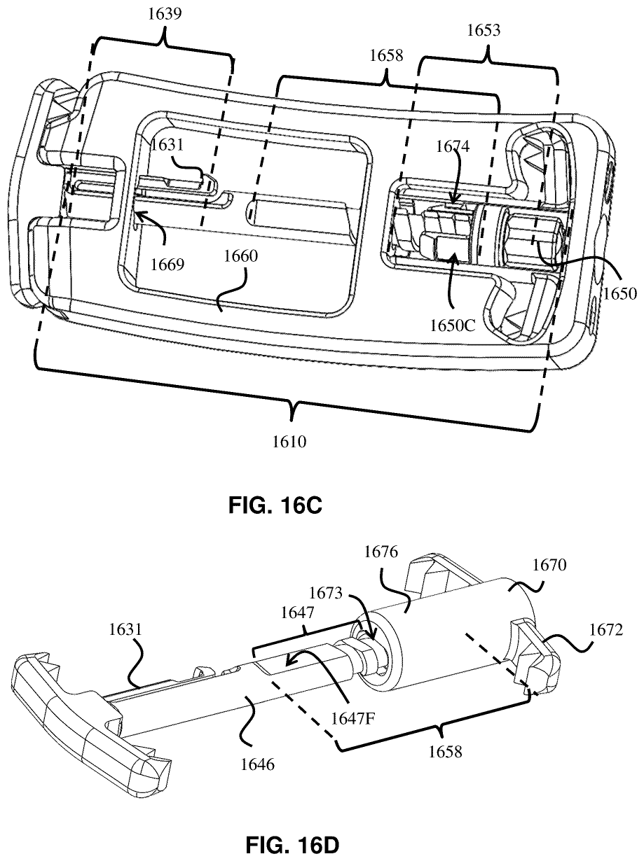

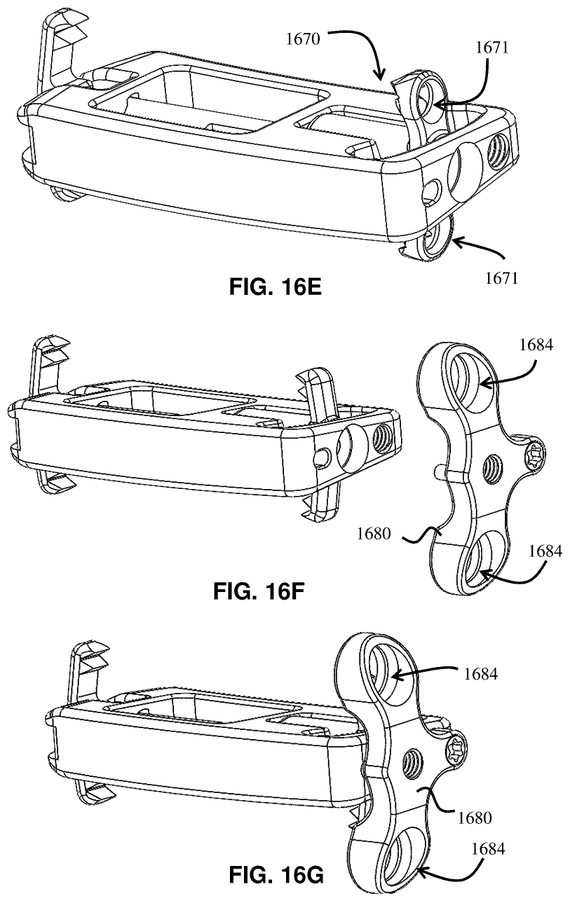

As with the other embodiments, the example implant device shown in FIGS. 16A-16G comprises a cage, one or more staple and a coupling mechanism providing a means to manipulate the one or more staple to secure the cage to a bone.

Referring to FIG. 16A, the one or more staple comprises a staple with a staple shaft 1646 and a distal staple head 1642. The staple shaft 1646 is configured to be engaged by the coupling mechanism to manipulate the staple shaft 1646 and staple head 1642. The coupling mechanism may be any element or combination of elements to couple the staple to the cage 1660 and allow the staple to be manipulated. As shown, the coupling mechanism 1653 comprises a nut 1650 configured to engage a coupling portion 1649 of the staple shaft. The nut 1650 is internally threaded nut 1650 with the threads of the nut 1650 configured to mate and engage with a coupling portion 1649 of the staple shaft 1646 whereby movement of the nut 1650 engages and moves the staple shaft 1646. The nut 1650 is configured to rotate within but be longitudinally constrained in a channel of the cage 1660. The nut 1650 is also configured to be engaged by an engagement tool such as a drive rod to move the nut 1650. As shown, the nut 1650 is configured to be constrained within but rotate relative to the cage 1660 and this longitudinally constrained rotation engages the threads of coupling portion 1649 of the staple shaft 1646. When the staple shaft 1646 is constrained from rotating, this engagement causes the staple shaft 1646 to translate. When the staple shaft 1646 is not constrained from rotating, the staple shaft 1646 rotates with the rotation of the nut 1650.

This embodiment has a keyway mechanism configured to influence the movement of the staple shaft in the cage and may comprise any configuration of element that influence the movement of the staple. The keyway may be any element or combination of elements that interface with the key to influence or constrain movement of the staple shaft and staple head. In the example embodiments shown, the keyway mechanism is configured to engage the staple whereby the staple shaft may be any or all of (i) constrained from rotating at some points of translation, (ii) able to translate, (iii) able to rotate at some points of translation, and (iv) stopped from translation at some point of translation.

In the example embodiment shown in FIGS. 16C and 16D, an example of the keyway mechanism 1639 comprises a key 1631 and a keyway 1669. The key 1631 is coupled or integral with a portion of the staple shaft 1646 and has an external profile with multiple external profile sections that engage the keyway in the cage. The keyway 1669 is generally a through-hole in the cage 1660 that receives the staple shaft 1646, engages the external profile of the key 1631, and provides translation and rotation stops of the key 1631 and staple shaft 1646 to influence movement of the staple shaft 1646.

FIGS. 16E-16G show embodiments of the implant device with additional components to provide additional fixation for posterior inserted implant devices. FIG. 16E shows an embodiment of the implant device with through-holes 1671 in the proximal staple 1670 to allow for bone screws to be used to further secure the implant device to the bone. FIGS. 16F and 16G show an embodiment with a lateral plate 1680 having through-holes 1684 to accept bone screws to be anchored in the bone. FIG. 16F shows the lateral plate 1680 separated from the cage and FIG. 16G shows the lateral plate 1680 secured to the implant device and ready to receive the bone screws. In addition to the single two-hole lateral plate shown, other embodiments may include two plates on both sides of the vertebral body may be used with screws to provide additional fixation.

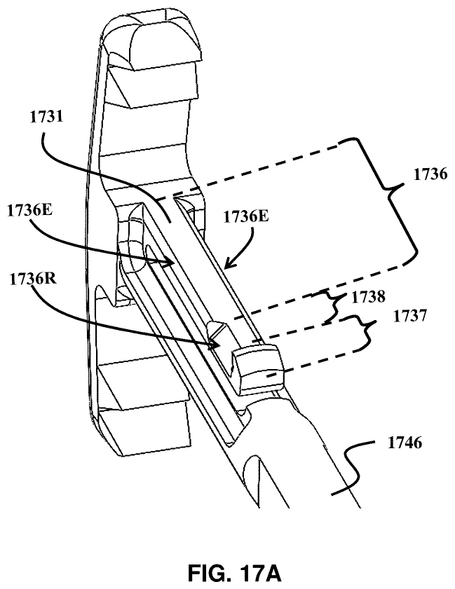

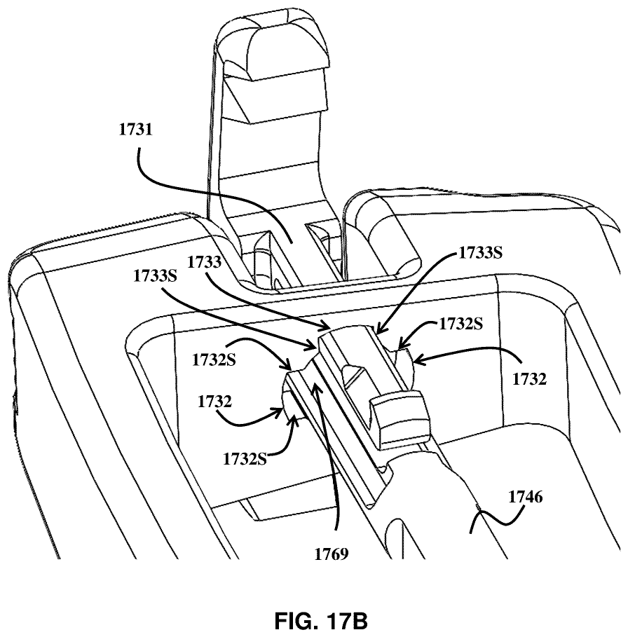

FIG. 17A shows details of a key and FIG. 17B shows details of the key engaged with the keyway. As shown, the key 1731 may have a rotation constraining portion 1736, a translation stop portion 1737 and a rotation allowing portion 1738. The key 1731 is configured to be received in the keyway 1769 and have its external profile engage the internal surface of the keyway 1769. For example, the key 1731 may be a flexible prong biased to push the external surface of the key 1731 against the internal surface of the keyway 1769. The keyway 1769 may also have multiple features to engage the key 1731 to interface with an influence and constrain movement of the staple shaft 1746 and staple head. For example, the keyway 1769 may have a surface profile defining different portions of the keyway 1769. For example, the surface profile may have notches that define different portions and features of the keyway 1769. For example, the keyway 1769 may have a rotation constraining portion 1732 having rotation stops 1732S whereby when the key is in the rotation constraining portion 1732 rotation of the staple shaft is constrained from rotating by the rotation stops 1732S. The keyway 1769 may have a deployment position portion 1733 having rotation stops 1733S whereby when the key 1731 is in the deployment position portion 1733, the staple shaft 1746 is constrained from rotating by the rotation stops 1733A and the staple head is positioned in the deployed position.

The rotation constraining portion 1736 of the key 1731 may be any method of stopping translation of the key 1731 and the staple shaft 1746. In the example shown, the rotation constraining portion 1736 of the key 1731 is a section with a cross-sectional profile having edges 1736E that engage rotation stops in the keyway 1769 that influence the rotation of the key 1731 and the staple shaft 1746. For example, the edges 1736E of the rotation constraining portion 1736 of the key 1731 engage the rotation stops 1733S in the rotation constraining portion in the keyway such that the while the staple shaft 1746 and the key 1731 translate with this portion of the key 1731 engaging the keyway 1769, the staple shaft 1746 is retrained from rotating. The translation stop portion 1737 of the key 1731 may be any method of stopping translation of the key 1731 and the staple shaft 1746. For example, the translation stop portion 1737 may comprise a protrusion on the key 1731 that engages a sidewall of the cage around the keyway 1769 preventing the staple shaft 1746 from further translation. The rotation allowing portion 1738 of the key 1731 may be any shape of the key 1731 that allows rotation of the key 1731 and the staple shaft 1746 past the rotation stops of the keyway 1769. For example, the rotation allowing portion 1738 of the key 1731 may comprise a portion of the key 1731 with a cross-sectional profile having a reduced edge 1736R that allows the key 1731 to flex to allow the key 1731 and the staple shaft 1746 to rotate through the rotation constraining portion 1732 and into the deployment position portion 1733. The flex of the key 1731 generally urges the key 1731 to be in the deployed position notch. Generally, the cross-sectional profile of the key 1731 at the rotation allowing portion 1738 only has one reduced edge 1736R that allows rotation which generally only allows rotation in one direction. The edge 1736E continuing on the other side of the key 1731, constrains rotation of the key 1731 and the staple shaft 1746 in the other direction which keeps the key 1731 and the staple shaft 1746 in the deployed position when the staple shaft 1746 is rotated in the other direction to translate the staple shaft 1746 in the other direction.

Also shown in FIGS. 16A and 16B is a second, proximal staple 1670. The proximal staple 1670 has a staple shaft, here barrel 1676 with a through-hole, configured to receive the nut 1650 and the proximal end of staple shaft 1646. The barrel 1676 and the nut 1650 are configured to restrain the longitudinal movement of the nut 1650 but allow it to rotate. The constraining of the nut 1650 may be provided by any method that allows for the constrained movement of the nut.

In the example embodiment shown in FIG. 16C, the constraining of the nut 1650 is with a channel portion 1650C around the exterior surface of the nut 1650 that engages an internal rib 1674 in the proximal staple barrel 1676. The internal rib 1674 allows the nut 1650 to rotate but constrains its longitudinal positioning.

This example embodiment also has an engagement mechanism configured to further manipulate the proximal staple 1670. The engagement mechanism may be any configuration of implant device components or any combination of features of components that allow the rotation of the proximal staple 1670. In this example embodiment, as shown in FIG. 16D, the engagement mechanism 1658 comprises an engagement portion 1647 of the staple shaft 1646, which is one or more flat section 1647F of the exterior surface profile of the staple shaft 1646. This engagement portion 1647 is configured to mate with a mating engagement portion 1673 of the barrel 1676. The engagement portion 1673 of the barrel may be any method of allowing the barrel 1676 to manipulate the staple shaft 1646 and the proximal staple head 1672. As shown, the engagement portion 1673 of the barrel 1676 is a mating flat surface profile in the distal end of the barrel 1676 that mates with the engagement portion 1647F of the staple shaft 1646 to allow a rotation of the staple shaft 1646 to also rotate the barrel 1676 and rotate the proximal staple 1670. The engagement portion 1647F extends along a portion of the staple shaft 1646. This allows the staple shaft 1646 to translate relative to the proximal staple 1670 while maintaining the relative alignment of the staple shaft 1646 and the proximal staple 1670.

FIG. 16C shows an example of the coupling mechanism 1610 which generally includes the components, features of the components, and the interfaces of the components to influence the manipulation of the implant device components such as the staple. In this embodiment, the coupling mechanism 1610 generally includes the coupling mechanism 1653, the components of the engagement mechanism 1658 and the components of the keyway mechanism 1639. It is understood that in different embodiments, the coupling mechanism 1610 may comprise any number of and any combination of these elements.

Operationally, referring to the example embodiment of FIGS. 16A-16D, the implant device is inserted in the insertion position with the distal staple 1640 and the proximal staple 1670 generally parallel to each other and within the upper and lower surfaces of the cage 1660. The nut 1650 is rotated which engages the staple threads of the coupling portion 1649 of the staple shaft 1646. Initially, the rotation constraining portion of the key 1631 engages the rotation constraining portion of the keyway 1669 and the staple shaft 1646 translates in one direction. As the nut 1650 is rotated, the staple shaft 1646 and the key 1631 translate until the translation stop hits the keyway 1669 and translation is stopped. In this position, the rotation allowing portion of the key 1631 allows the staple shaft 1646 to rotate and the key 1631 is then constrained in the deployment position notch of the keyway 1669. In this position, the staple head 1642 is generally in the deployed position. When the staple shaft 1646 rotates, the engagement mechanism 1658 also rotates the proximal staple 1670. Generally, the engagement portion 1647 engages engagement portion 1673 to rotate the proximal staple 1670 with the distal staple 1640. With the staple shaft 1646 and staples in this position, the nut 1650 can be rotated in the other direction. As the nut 1650 rotates in the opposite direction, it engages the staple shaft 1646 and the staple shaft 1646 is translated in the other direction. As the staple shaft is translated, the key translates through the keyway and the rotation constraining portion of the key 1631 is again engaged with the deployment position portion of the keyway 1669. The cross-sectional profile of the key 1631 in the deployment position portion of the keyway 1669 prevents rotation of the key 1631 and both staples 1640 and 1670 stay in the deployed position during this translation.

Suitable tools to engage the nut and position the implant device may be the insertion handle assemblies and staple drive handle assemblies as described herein.

### Rotating Staple and Stable Plate Embodiment

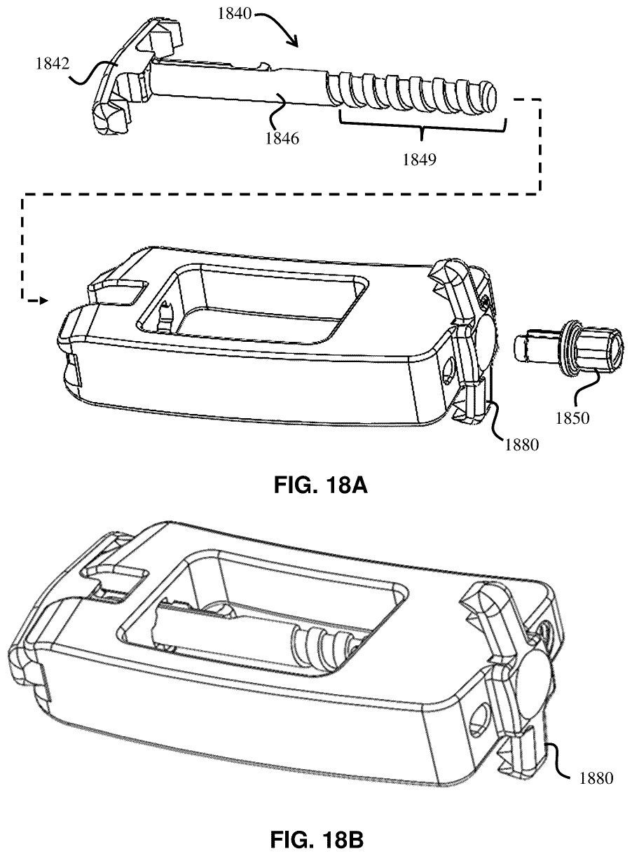

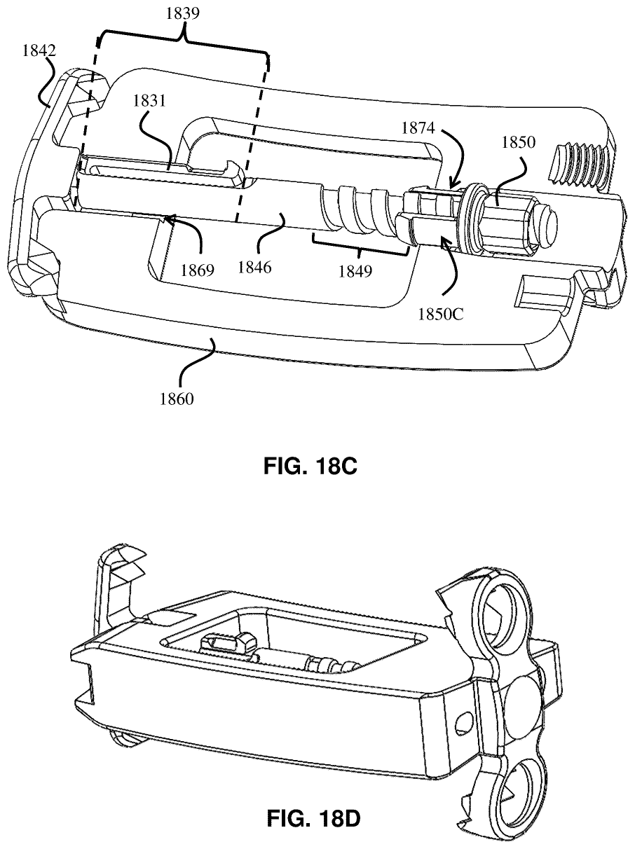

As with the other embodiments, the example implant device illustrated in FIGS. 18A-18D comprises a cage, one or more staple and a coupling mechanism providing a means to manipulate the one or more staple to secure the cage to a bone.

Referring the embodiment of FIG. 18A, the one or more staple comprises a staple 1840 with a staple shaft 1846 and a distal staple head 1842. The staple shaft 1846 is configured to be engaged by a coupling mechanism to manipulate the staple shaft 1846 and staple head 1842. As shown, the coupling mechanism comprises an internally threaded nut 1850. The threads of the nut 1850 are configured to mate and engage with a coupling portion 1849 of the staple shaft 1846 whereby movement of the nut 1850 engages and moves the staple shaft 1846. The nut 1850 is also configured to be engaged by an engagement tool such as a drive rod to move the nut 1850. The nut 1850 is configured to rotate within but be longitudinally constrained in a channel of the cage.

FIG. 18C shows a view of the implant device with a cut-away section of the cage 1860 showing details of the coupling mechanism. As shown, the nut 1850 is configured to be constrained within but rotate in the channel of the cage 1860. The constraining of the nut 1850 is with a channel portion 1850C around the exterior surface of the nut 1850 that engages an internal rib 1874 in the cage 1860. The internal rib 1874 allows the nut 1850 to rotate but constrains its longitudinal positioning. The longitudinally constrained rotation engages the threads of coupling portion 1849 of the staple shaft. When the staple shaft 1846 is constrained from rotating, this engagement causes the staple shaft 1846 to translate. When the staple shaft 1846 is not constrained from rotating, or when translation is constrained and rotation is not constrained, the staple shaft 1846 rotates with the rotation of the nut 1850.

The coupling mechanism of this embodiment also includes a keyway mechanism that has similar components and functions similar to the embodiment shown in FIGS. 16A-16G. The keyway mechanism is configured to engage the staple whereby the staple shaft may be any or all of (i) constrained from rotating at some points of translation, (ii) able to translate, (iii) able to rotate at some points of translation, and (iv) stopped from translation at some point of translation. The keyway mechanism 1839 comprises a key 1831 and a keyway 1869. The key 1831 is coupled or integral with a portion of the staple shaft 1846 and has an external profile with multiple external profile sections that engage the keyway 1869 in the cage 1860. The keyway 1869 is generally a through-hole in the cage 1860 that receives the staple shaft 1846, engages the external profile of the key 1831, and provides translation and rotation stops of the key 1831 and staple shaft 1846 to influence movement of the staple shaft 1846.

This embodiment also has a proximal staple, or a rigid plate that does not rotate with the staple shaft. As shown in FIG. 18A, the proximal staple generally has one or more proximal staple tine extending above in a non-parallel orientation to the upper surface of the cage 1860 and one or more proximal staple tine extending below and in a non-parallel orientation to the lower surface of the cage 1860.

As shown in FIG. 18D, it is understood that the proximal staple may be a plate or other anchoring element configured to help secure the implant device to the bone, and the proximal staple may be removable and secured to the implant device before or after implanting. FIG. 18D also shows an embodiment of the implant device with through-holes in the staple to allow bone screws to be used to further secure the implant device to the bone.

Operationally, this embodiment functions similar to the example embodiment of FIGS. 16A-16E with an exception of the operation of the engagement mechanism and the rotation of the proximal staple. The implant device is inserted with the distal staple 1840 and the distal staple head 1842 in the insertion position. The nut 1850 is rotated which moves the distal staple 1840 and distal staple head 1842 from the insertion position to the extended position. Continued rotation of the nut 1850 moves the distal staple 1840 and the distal staple head 1842 from the extended position and further rotation moves the distal staple 1840 and the distal staple head 1842 to the stabilized position. The distal staple 1840 and the distal staple head 1842 are retracted towards the cage 1860 and the plate 1880 and the implant device is secured to the bone.

Suitable tools to engage the nut and position the implant device may be the insertion handle assemblies and staple drive handle assemblies as described herein.

### Bi-lateral Implant Device

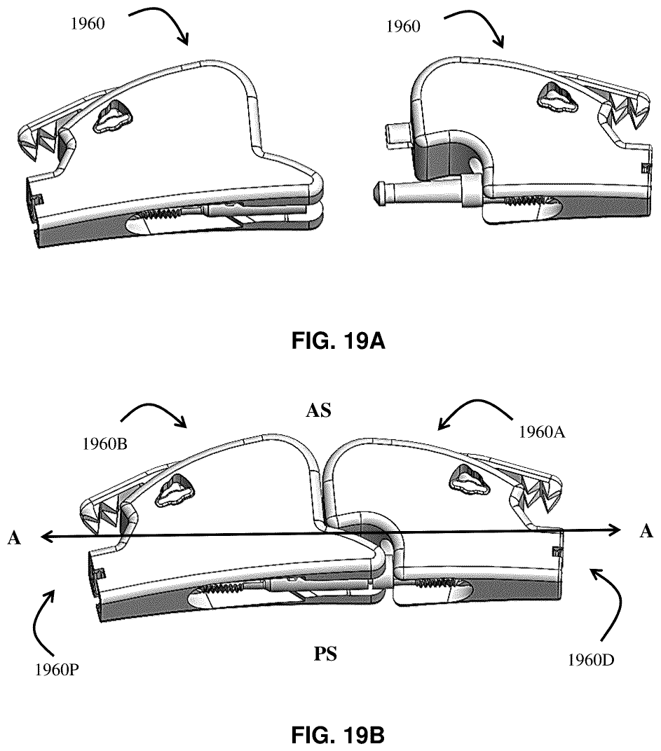

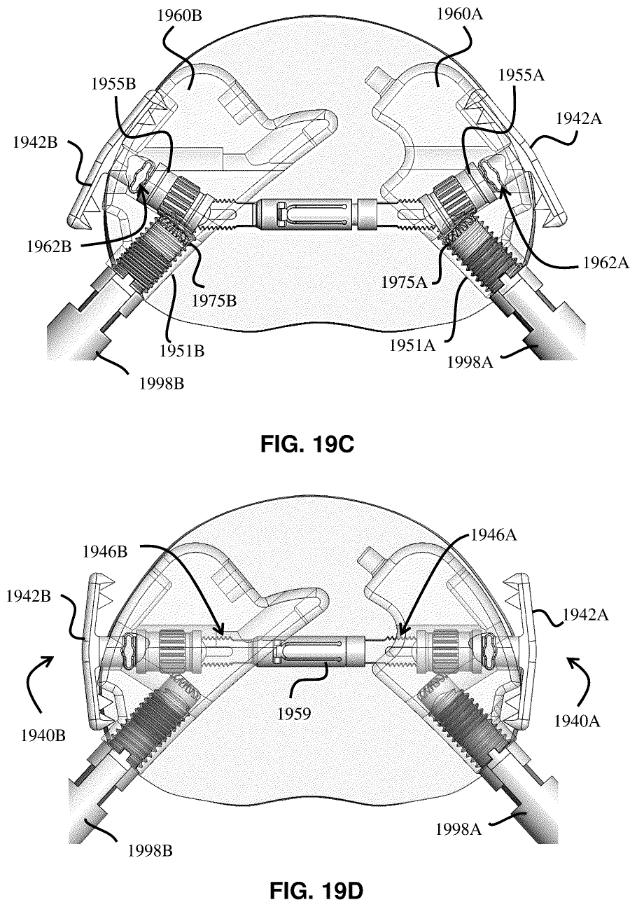

FIGS. 19A-19D show another example embodiment of an implant device being a bi-lateral implant device with the cage 1960 comprising multiple cage sections or portions 1960A and 1960B. As with the other embodiments, the implant device comprises a cage 1960, one or more staple and a means to manipulate the one or more staple. The illustrated means to manipulate the staple is with a coupling mechanism that is engaged by a tool such as a drive rod.

In this embodiment, the cage 1960 comprises a first section 1960A and a second section 1960B, each with their own staple head and coupling mechanism. The two cage sections 1960A and 1960B may have different dimensions to accommodate anatomical differences or to help correct the alignment of the spine. FIG. 19A shows the implant device with the two cage sections 1960A and 1960B unconnected and FIG. 19B shows the implant device with the two cage sections 1960A and 1960B connected. The cage 1960 has a posterior side PS and an anterior side AS on either side of a longitudinal midline A of the cage 1960 extending from the proximal end 1960P to the distal end 1960D of the cage. The anterior side AS of the implant device and the cage sections 1960A and 1960B is shown and is configured to be positioned in an anterior orientation to the body when the orthopedic implant device is implanted. The posterior side PS of the cage 1960 is configured to be positioned in a posterior orientation to a mammalian body when the orthopedic implant device is implanted in the mammalian body.

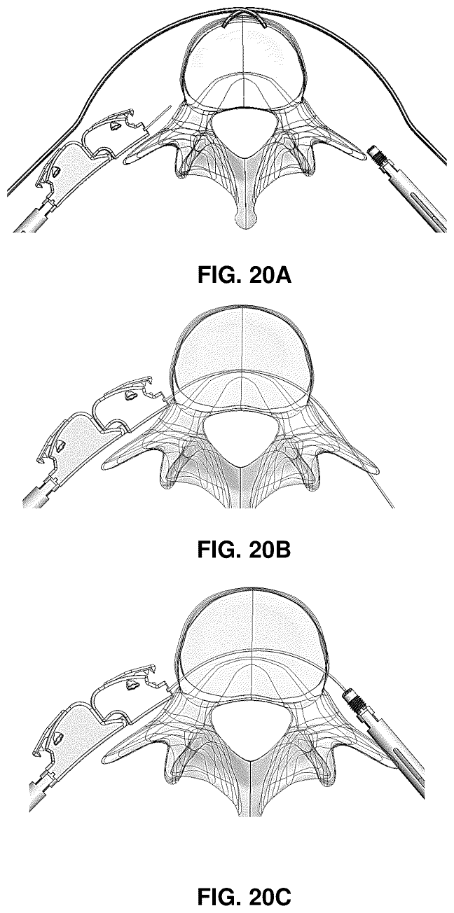

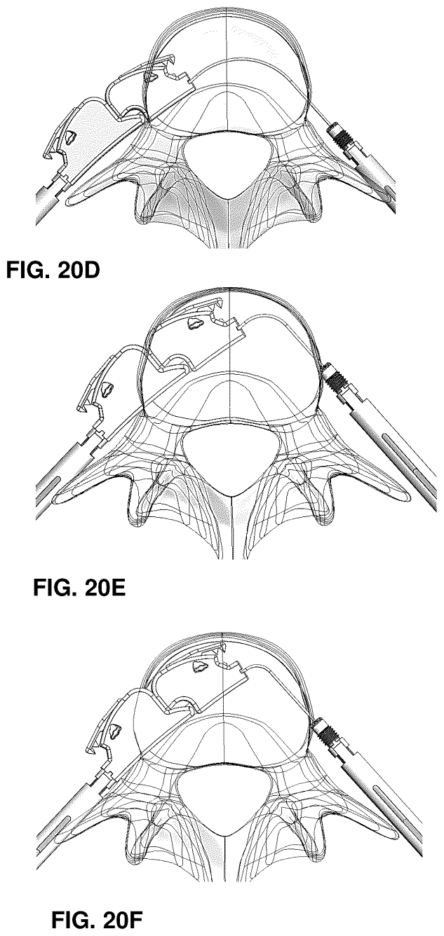

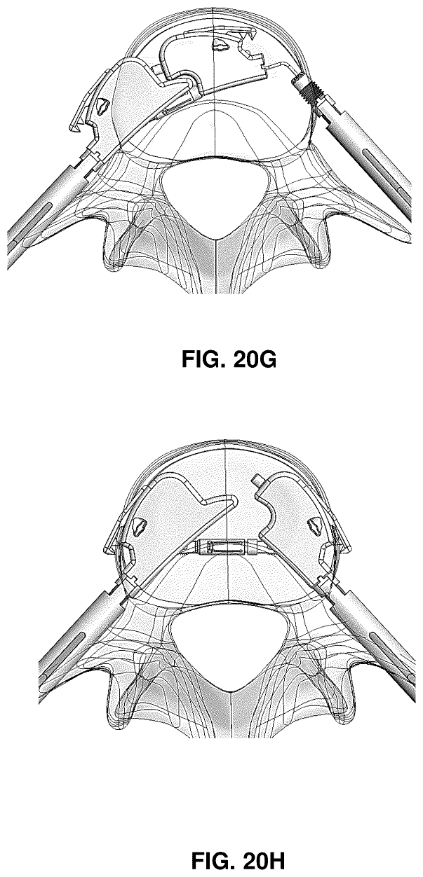

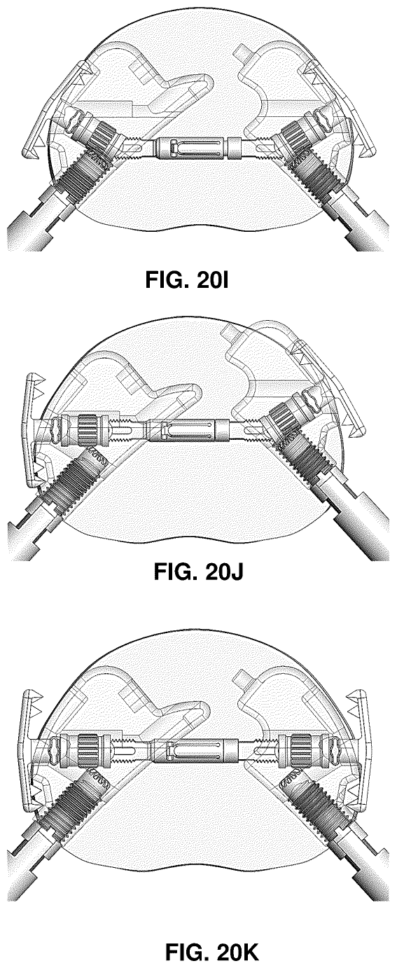

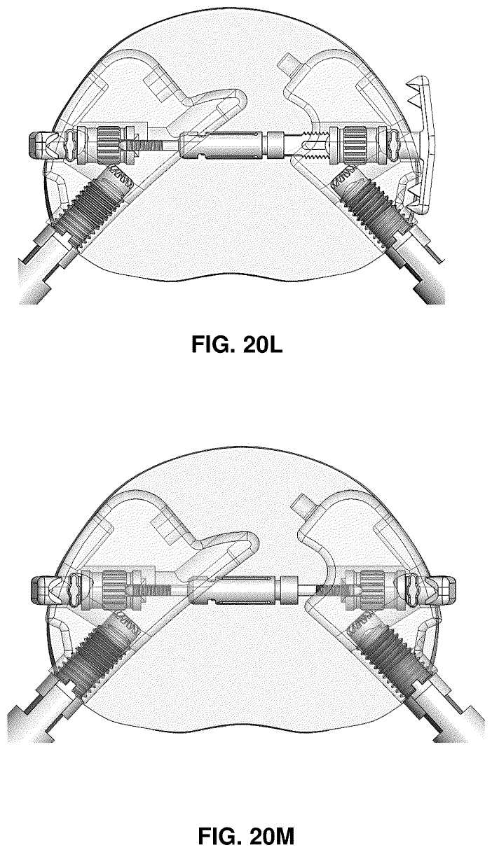

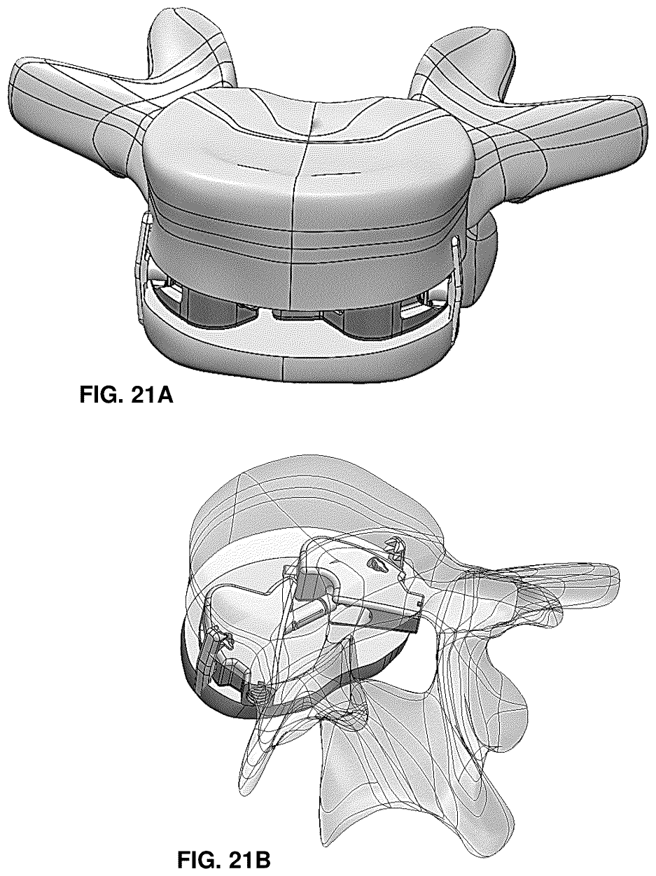

FIG. 19C shows the cage sections 1960A and 1960B positioned in an osteotomy space of a vertebral body with the staples 1942A and 1942B in an insertion position and illustrates the coupling mechanism in this embodiment. The coupling mechanism generally comprises drive couplers 1951A and 1951B to receive the end of two drive rods 1998A and 1998B and drive cylinders 1955A and 1955B that are coupled with the staples and directly engaged by the drive rods 1998A and 1998B. As with other embodiments, the drive cylinders 1955A and 1955B have teeth around their outer surface to engage teeth 1975A and 1975B on the end of the drive rods 1998A and 1998B so that a rotation of the drive rods rotates the drive cylinders. The drive cylinders 1955A and 1955B are configured to engage the staple heads 1942 A and 1942B when rotated in one direction. Rotation in this direction rotates the staple heads 1942A and 1942B and positions them in the deployed position (see FIG. 20M). The drive cylinders 1955A and 1955B may also be internally threaded to mate and engage with the threaded exteriors of the staple shafts 1946A and 1946B so that a rotation of the drive cylinders 1955A and 1955B engages the staple shafts 1946A and 1946B and causes them to move. The staples 1940A and 1940B are also pivotally coupled to the cages with pivots 1962A and 1962B so that the drive cylinders 1955A and 1955B can be pushed by the drive rods 1998A and 1998B and the staples 1940A and 1940B and staple heads 1942A and 1942B pivot and move from the insertion position to the extended position.

FIG. 19D shows the cage sections 1960A and 1960B with the drive cylinders 1955A and 1955B aligned and the staple heads 1942A and 1942B in the extended position. The staples 1940A and 1940B are moved to this position by pushing the drive rods 1998A and 1998B forward and forcing the staples to pivot. The extended position in this embodiment is slightly different than the extended position of the other embodiments. As shown, the staples 1940A and 1940B do not translate away from each other, but the staple tines of the staple heads 1942A and 1942B do generally get positioned so that they both extend outside of the vertebral body walls. The staples 1940A and 1940B are pushed by the drive rods 1998A and 1998B and pivot to generally have the connecting mechanism 1959 and the two staple shafts 1946A and 1946B align and move the staple heads 1942A and 1942B accordingly. In this embodiment, the connection mechanism 1959 is another component of the coupling mechanism. In this position, the threaded ends of the staple shafts 1946A and 1946B are aligned with and configured to rotate the drive cylinders 1955A and 1955B. The drive cylinders 1955A and 1955B are rotated in one direction to position the staple heads 1942A and 1942B in the deployed position. The rotation of the staple heads 1942A and 1942B may be limited by a rotational stop. The drive cylinders 1955A and 1955B are rotated in the other direction by the drive rods 1998A and 1998B where the internal threads of the drive cylinders engage the staple shafts 1946A and 1946B and the staple heads 1942A and 1942B retract toward each other. This retraction positions the staples 1940A and 1940B and the staple heads 1942A and 1942B in the stabilized position.

As shown in FIG. 19D, the drive rod rotation axis and the drive coupler rotation axis are non-parallel and not aligned with the staple rotation axis or the staple translation axis.

### Other Embodiments of a Bi-lateral Implant Device

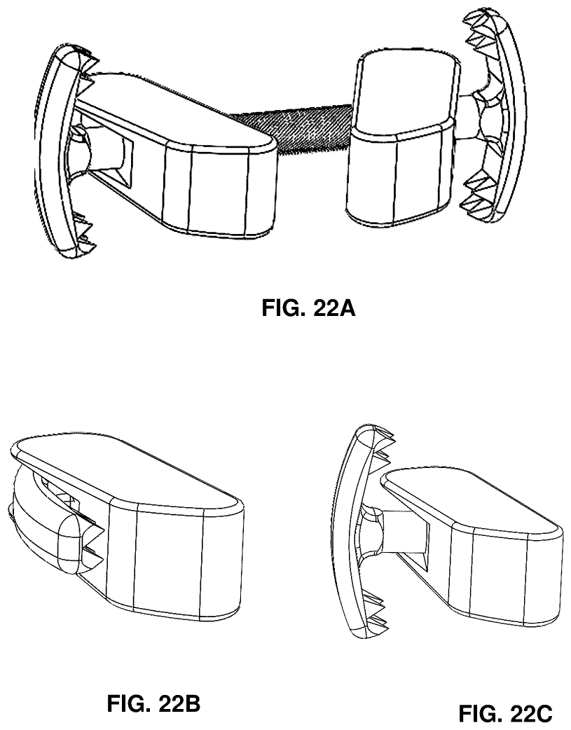

FIGS. 22A-22C show another example embodiment of an implant device. This embodiment is a bi-lateral device with the cage comprising multiple cage sections. In the embodiment show, the cage comprises two cage sections. FIG. 22A shows the implant device with the staples in a deployed position. FIG. 22B shows one of the cage sections with the staple in the insertion position and FIG. 22C shows one of the cage sections with the staple in the deployed position.

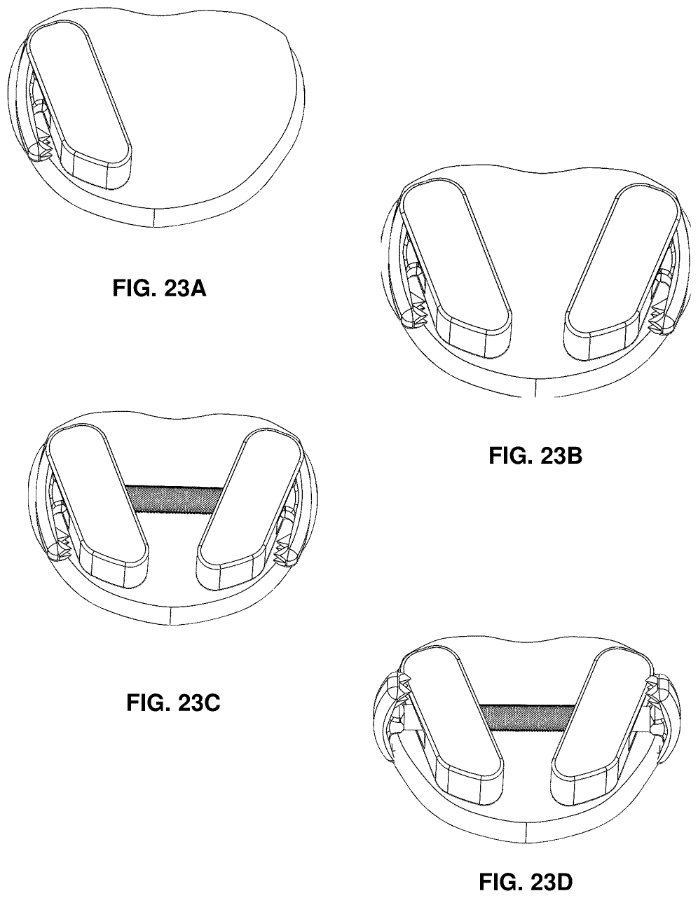

FIGS. 23A-23D show different views of this example embodiment as positioned on a vertebral body portion. FIG. 23A shows one of the cage sections positioned on the vertebral body portion with the staple in the insertion position. FIG. 23B shows two cage sections with both staples in the insertion position. FIG. 23C shows two cage sections coupled with a coupling element such as a cord, cable, tether, or other coupler and the staple are in the insertion position. FIG. 23D shows two cage sections coupled with the staples in the deployed position.

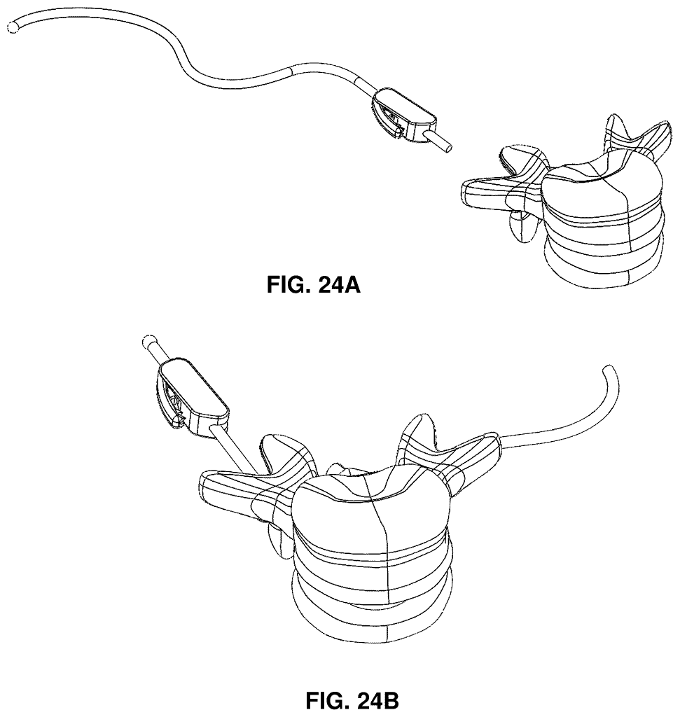

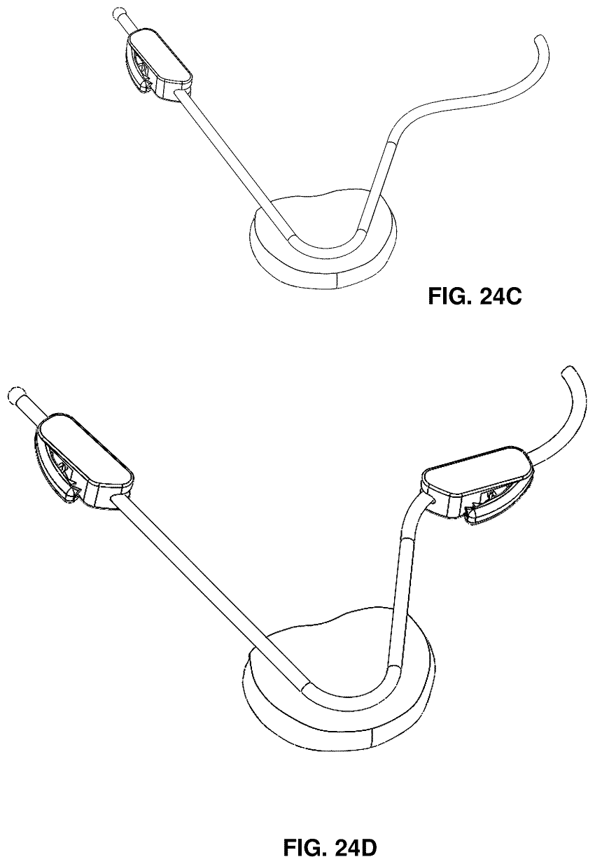

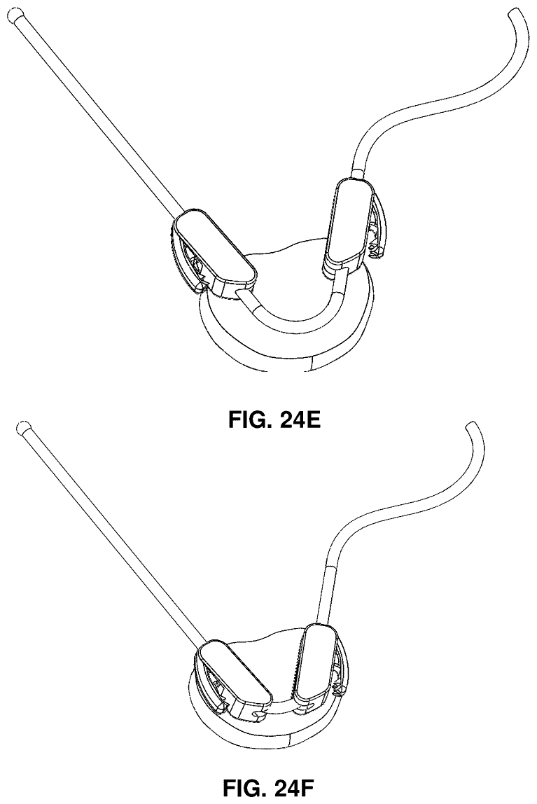

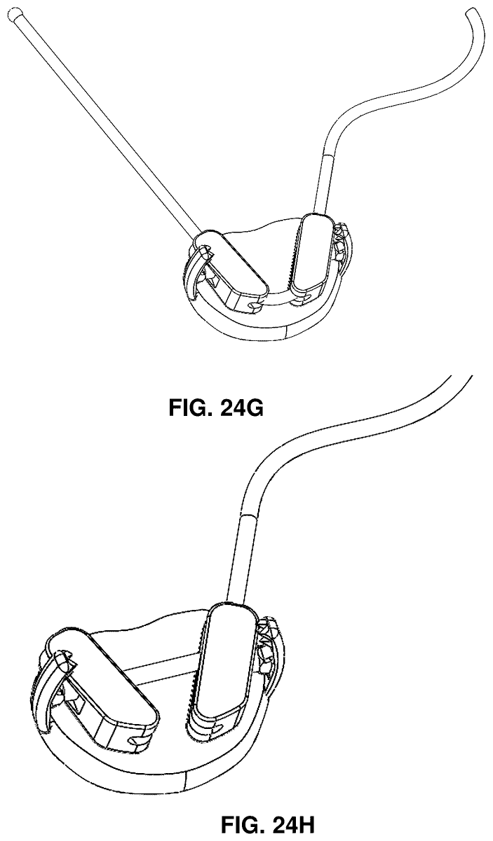

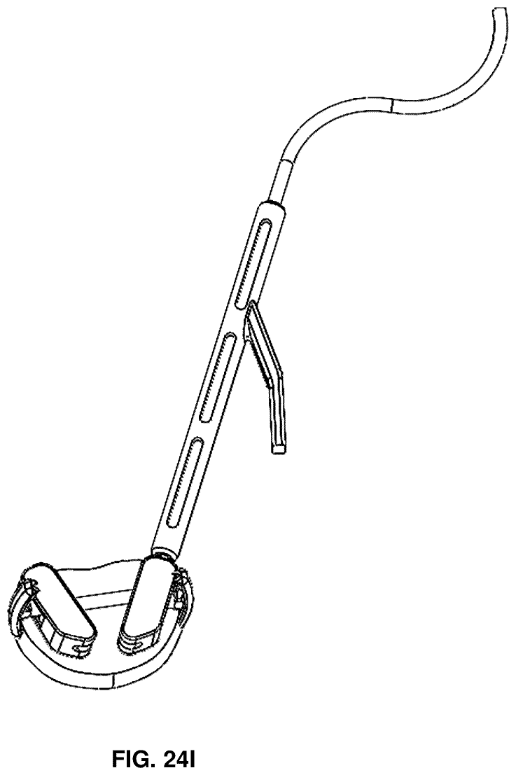

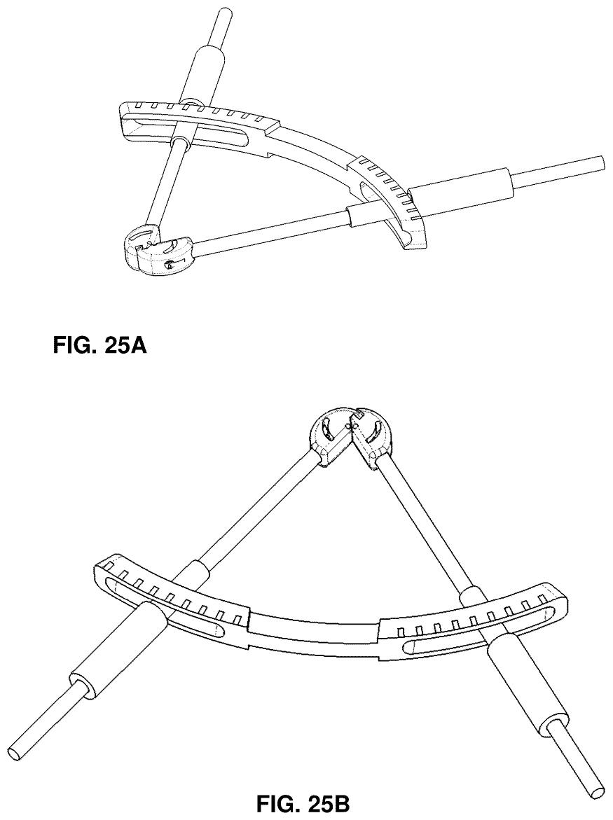

FIGS. 24A-24I show details of an example embodiment of positioning and actuating this implant device. FIG. 24A shows one of the cage sections with a cable or flexible tether extending through the body of the cage. The cable/tether may have a ball on its end to prevent the implant from slipping off the cable. FIG. 24B shows the positioning of the cable/tether through the vertebral body and having the far end extend through the vertebral body's other side. FIG. 24C shows the example of FIG. 24B with the top vertebra portion hidden for clarity. FIG. 24D shows the feeding of the far end of the cable/tether through the second cage sections. FIGS. 24E and 24F shows the positioning of both cage sections onto the vertebral body portion. In this position, the staples are rotated by the insertion handle assembly. FIG. 24G shows the staples in a deployed position. FIG. 24H shows the cable/tether being retracted by pulling its far end until the ball on the near end of the cable/tether is stopped in the first cage section. In FIG. 24I, the cable/tether is tensioned using a tensioning instrument which pulls the staples together, retracting them to a stabilized position with the staples secured in the side wall of the bone. The far end of the cable/tether is then cut.

### Other Embodiments of a Bi-lateral Implant Device

FIGS. 25A and 25B show different views of another example embodiment of an implant device together with an insertion handle assembly. FIGS. 25A and 25B show different views of an implant device with two cage sections coupled to an insertion handle assembly.

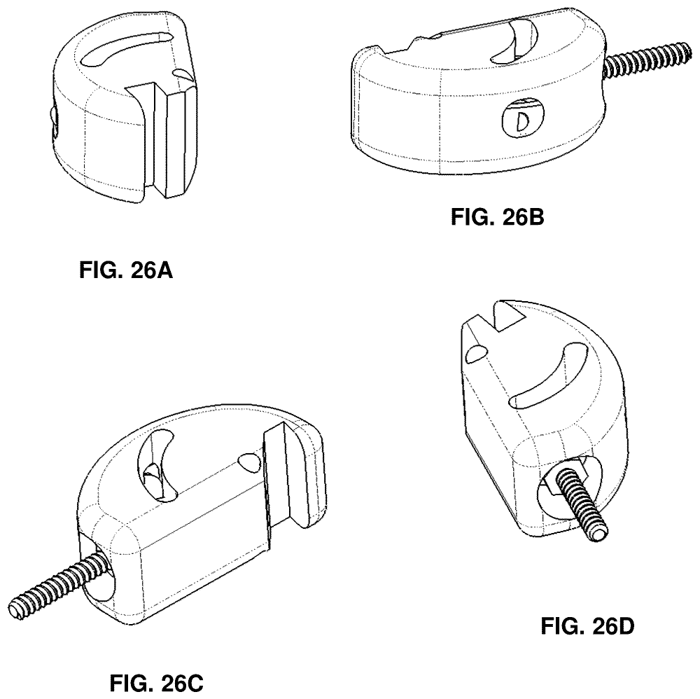

FIGS. 26A-26D show different views of one cage section of this example embodiment. FIG. 26A shows a top anterior perspective view, FIG. 26B shows a top side perspective view, FIG. 26C shows another top side perspective view and FIG. 26D shows a top posterior perspective view. As shown, the cage section has a coupling mechanism. In this example, the coupling mechanism includes a threaded rod received in a threaded nut.

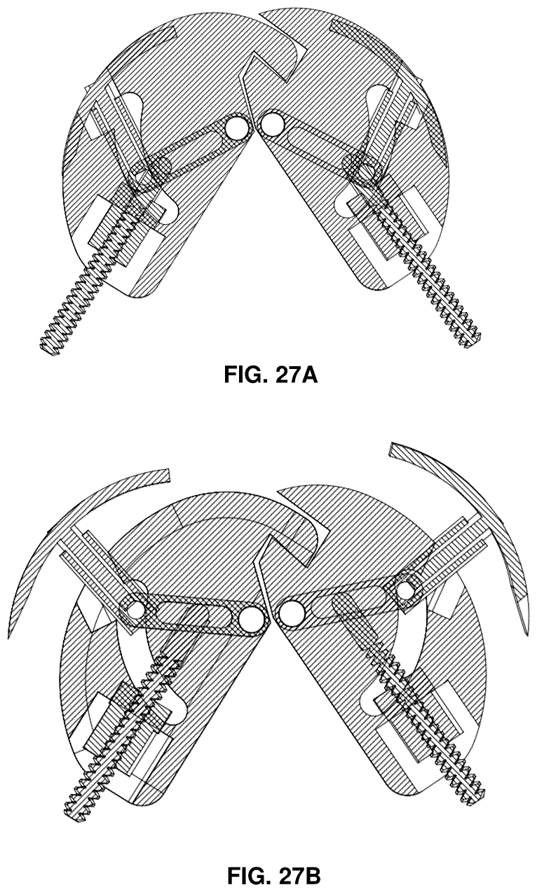

FIGS. 27A and 27B show additional details of the coupling mechanism and actuating this implant device. FIG. 27A shows two cage sections, each with a coupling mechanism. In this example, the coupling mechanism comprises a threaded rod received in a threaded nut. In this example, the nut is turned which moves the threaded rod to turn and actuates the hinged pivot drive. As the nut is turned, the threaded rod extends and pushes the hinged pivot drive to extend the staple out of the cage. As the staple extends, it is rotated by a slotted pin that engages with the cage body. FIG. 27B shows the hinged pivot drive extended, and the staples extended and rotated into the deployed position.

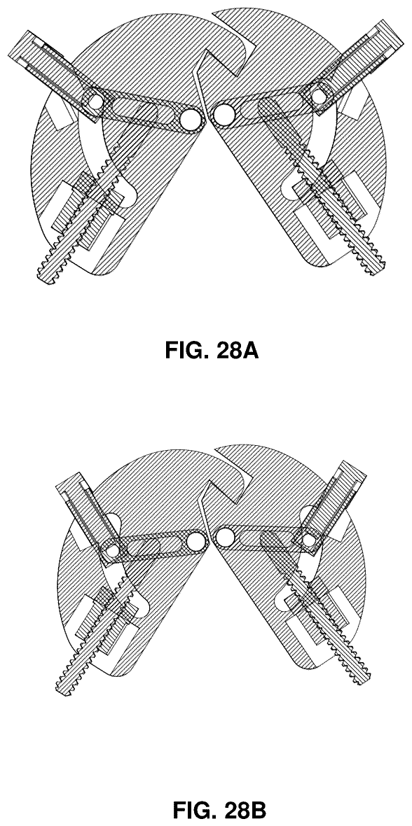

FIGS. 28A and 28B show additional details of actuating this implant device. FIG. 28A shows the retraction of the staples by turning the nut in the opposite direction to pull the threaded rod and hinged pivot drive. FIG. 28B shows the retracting of the staples to secure the staples to the side walls of the bone and lock the two cage sections together.

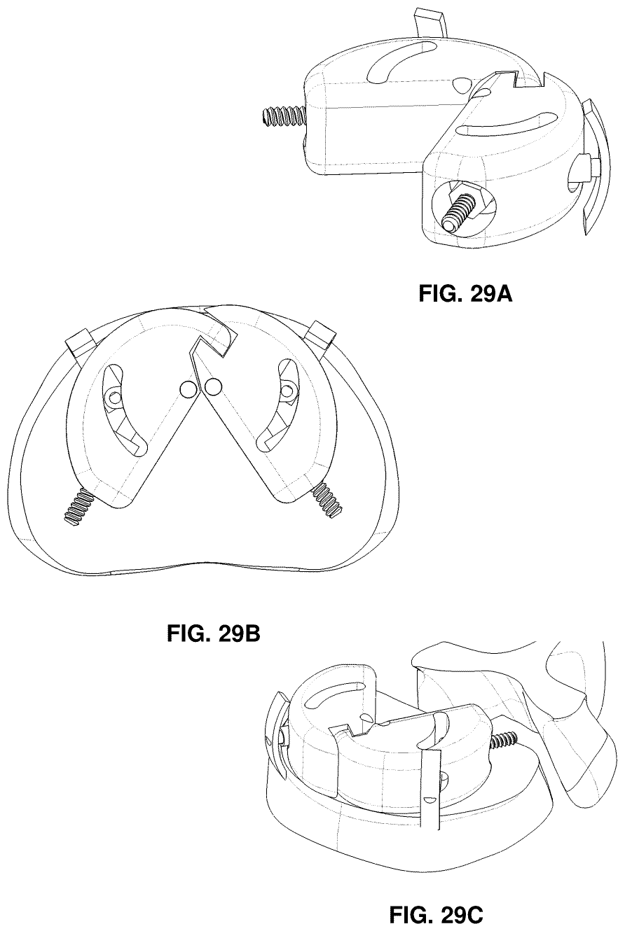

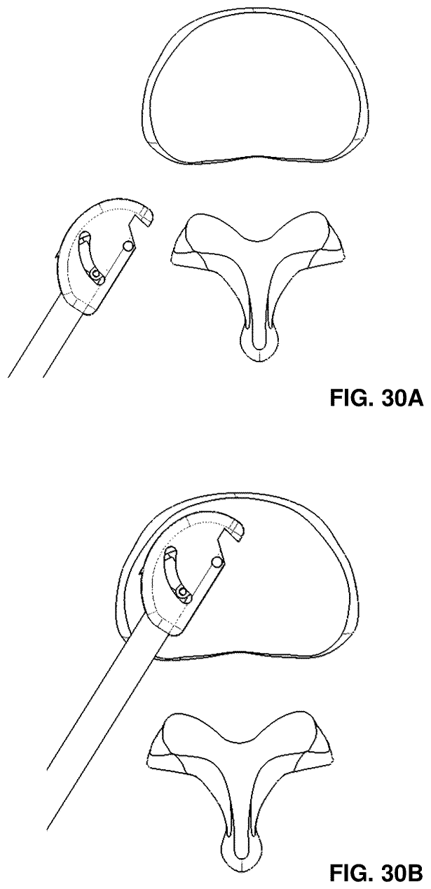

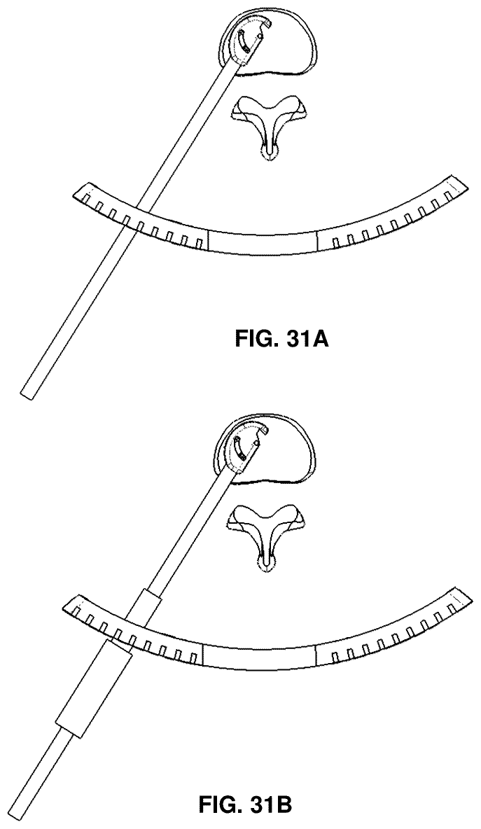

FIGS. 29A-29C show views of this example embodiment of the implant device. FIG. 29A shows the implant device locked together with the staples in the stabilized position. FIG. 29B shows a top view of the implant device secured to the side wall of the vertebral body and FIG. 29C shows a similar perspective view.

FIGS. 30A and 30B show examples of positioning this example embodiment of the implant device with an insertion handle assembly. FIG. 30A shows the implant being positioned from a posterior lateral position. FIG. 30B shows the implant positioned on the vertebral body.

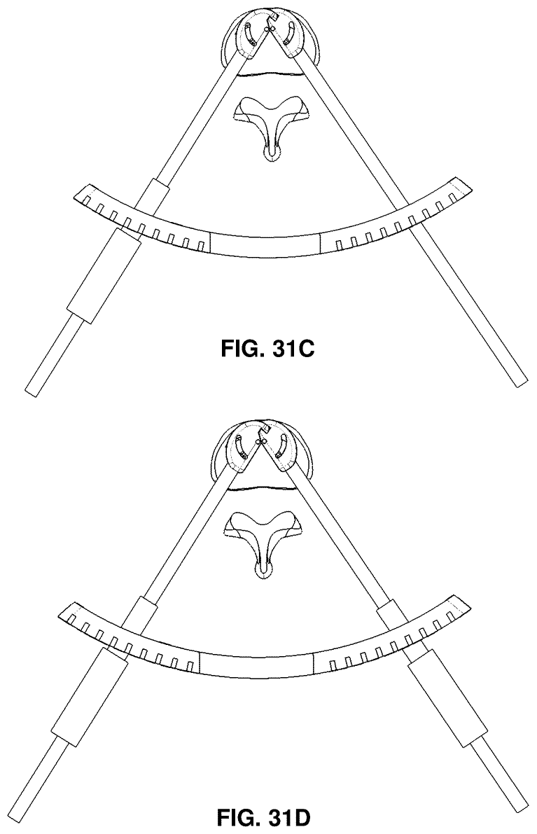

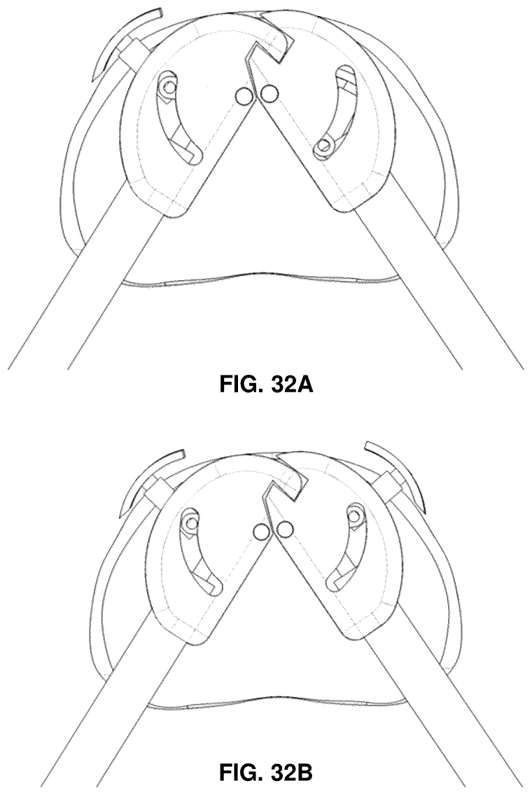

FIGS. 31A-31D show additional views of positioning this example embodiment int the implant device. FIGS. 31A and 31B show the first cage section of the implant device positioned with an implant guide tool and the insertion handle assembly. FIGS. 31C and 31C show the second cage section of the implant device positioned with an implant guide tool and the insertion handle assembly.

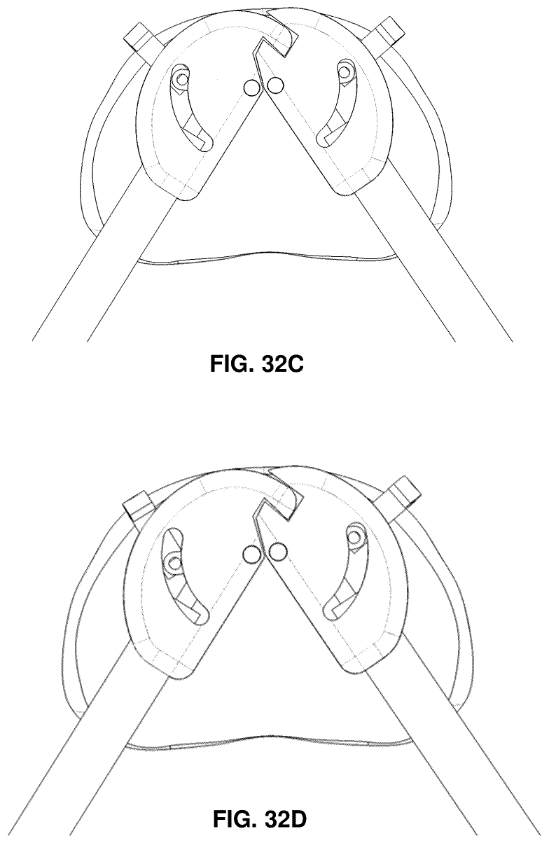

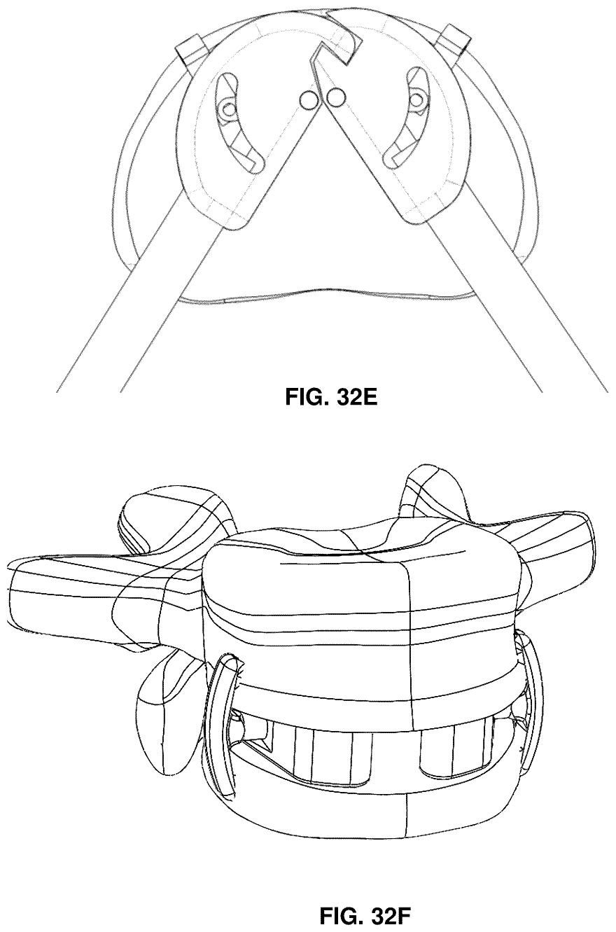

FIGS. 32A-32F show additional details of actuating this embodiment of the implant device. FIG. 32A shows two cage sections with one staple in an extended position. FIG. 32B shows two cage sections with both staples in an extended position. FIG. 32C shows two cage sections with both staples rotated in a deployed position. FIG. 32D shows a top view of two cage sections with one staple in a retracted and stabilized position. FIG. 32E shows a top view of two cage sections with both staples in a retracted and stabilized position. FIG. 32F shows a perspective view of the implant device secured to the side wall of the vertebral body in a stabilized position.

In some embodiments, the cage may have a surface treatment or a lattice configuration on one of or both the upper surface of the lower surface to encourage bone growth, apposition, and/or adhesion.

When assembled and implanted in the vertebral body, the external surface dimension and configuration of the intravertebral implant device are able to correct the relative orientation of a superior endplate surface plane and an inferior endplate surface plane of a vertebral body to alter the alignment of the spine. The external surface configuration of the cage and the intravertebral implant device may be altered by using different configurations of intravertebral implant device components. For example, the cage may be configured to have different cage surface angles in either the coronal or sagittal planes to create different external surface configuration when implanted in the osteotomy. The cage may also be configured to have different heights to create different amounts of expansion when implanted in the osteotomy. Sets of multiple exchangeable cage configurations can provide implant device options to accommodate different vertebrae, different sized patients, different amounts of correction and different orientations of insertion.

Furthermore, some embodiments of the implant system may be configured to alter the alignment of the spine in multiple planes. This multi-plane alignment may be made by the insertion angle of the implant and/or the dimensions of the cage and the resulting cage surface angles.

In some embodiments, additional through holes may be provided in the cage to accommodate additional pedicle screws to further anchor the implant device to the vertebral body. In these embodiments, the pedicle screw may be received in additional through-holes in the implant device and into the vertebral body.

### The Implant System Used in Intervertebral Applications

The above-described implant systems may be used for intervertebral applications. For example, the implant systems may be able to use the cage to separate two vertebral bodies and the staples may be used to secure the implant device to the superior and inferior vertebral bodies. The intervertebral implant systems may be used for fusion of any vertebral bodies. For example, these intervertebral implant systems may be used in transforaminal lumbar interbody fusion (TLIF) surgery to replace intervertebral disks in a patient's lower back. In particular, embodiments of the implant system may be used at the L5-S1 level using the Wiltse approach trajectory.

### Application with External Staple Head Embodiments

To illustrate methods of using the disclosed implant system, the general steps used to perform a TLIF procedure with a posterior lateral approach as used for a posterior pedicle subtraction osteotomy using an embodiment of the implant device consistent with the embodiments of FIGS. 2A-2D will be described.

An implant device and insertion tools are provided and configured for insertion. An insertion handle assembly, a staple drive handle assembly and the implant device is provided and the insertion handle assembly and the staple drive handle assembly are coupled to the proximal end of the implant device.

An incision is made in the patient's back and a blunt dissection is performed from the incision to the spine to create an access portal to the spine.

Bony landmarks are identified such as the transverse processes and pedicles of the vertebral body.

The implant device is passed through the access portal from the posterior approach trajectory and positioned by pushing the device with the insertion handle. When the implant is positioned between the end plated of the two vertebral bodies, the staple drive handle is pulled retrograde and the insertion handle assembly pivots to allow the implant device to align laterally across the vertebral body.

With the drive rod of the staple drive handle assembly engaged with the coupling mechanism of the implant device, the drive rod is rotated by rotating knobs on the proximal ends of the staple drive handle assembly whereby the staples are extended, deployed, retracted, stabilized and secured against the side walls of the vertebral body.

It is understood that similar procedures may be used with the embodiment shown in FIGS. 11A-11D with one difference being that the staples are deployed and stabilized in the vertebral body by rotating and extending the staples.

It is also understood that similar methods may be used with the other embodiments of the vertebral implant system and may be used in intravertebral or arthrodesis applications.

For illustration purposes only, and not for limitation, embodiments of the implant system used for intervertebral applications are described and referred to as an implant system, a vertebral implant system, an intervertebral implant system, a vertebral implant device and an intervertebral implant device.

### Application with Bi-lateral Embodiments

To illustrate methods of using the disclosed implant system, the general steps used to perform a TLIF procedure with the Wiltse approach using an embodiment of the implant device consistent with the embodiments of FIGS. 19A-19D.

Two drive rods, a suture, a suture passer, and the implant device are provided. The implant and one drive rod are prepared by coupling the proximal end of the implant device to the drive rod with the drive coupler. The suture is connected to the distal end of the implant device as a guide suture.

Incisions are made on both sides of the patient's back creating an insertion side incision and a receiving side incision.

A blunt dissection is performed from the incisions to the spine to create an insertion side access portal and a receiving side access portal to the spine.

The suture passer is passed from the receiving side access portal to the insertion side access portal.

Both legs of the suture are threaded into the suture passer from the insertion side to the receiving side, and the suture passer is removed from the receiving side access portal passing both sutures from the insertion side to the receiving side.

The guide suture is passed through a second drive rod on the receiving side. The implant device is positioned by pushing the implant into the space from the insertion side with the drive rod and pulling on the guide suture to draw the second drive rod to the implant device.

The guide suture is also used to guide the second drive rod to the distal drive coupler where the end of the drive rod is coupled to the drive coupler.

With the second drive rod coupled, the guide suture can then be cut and removed by pulling it from the receiving side.

At this point, the device is ready to deploy the staples. With the drive rod of the staple drive handle assembly engaged with the coupling mechanism of the implant device, the drive rods are rotated by rotating knobs on the proximal ends of the drive rods and the staples are extended, deployed, retracted, stabilized and secured against the side walls of the vertebral body.

Appropriate instrumentation as known to a skilled artisan would be provided to the surgeon to assist and facilitate every step of the above implantation procedure. These instruments would include but not be limited to, cutting guides, cage introducer/retractor, cage inserter/holders, sizing template, drill template, drill bits, plate holder/introducer and screwdrivers. A skilled artisan would also adapt these instruments appropriately to accommodate the desired surgical approach trajectory such as posterior, posterior lateral or posterior oblique as may be used in a PLIF or TLIF procedure.

### The Implant System Used in Intravertebral Applications

It is understood that the above-described implant systems and methods may also be used for intravertebral applications. For example, the implant systems may be able to use the cage to separate two portions of a vertebral body after an osteotomy. In these procedures, the cage is positioned in the osteotomy space and the staples may be used to secure the implant device to the superior and inferior portion of the vertebral body.

Generally, these implant systems have similar features in respect to the horizontal plane so that sufficient structure is available to engage both portions of the vertebral body. These implant systems may also have retracting or expanding features for the staples to further secure the implant device to the walls of the vertebral bodies.

For illustration purposes only, and not for limitation, an example of the implant system used for intravertebral applications are described and referred to as an implant system, a vertebral implant system, an intravertebral implant system, a vertebral implant device and an intravertebral implant device.

### The Implant System Used in Arthrodesis Applications

As the above-described systems and devices may be configured for use in intervertebral or intravertebral applications, and the implant systems may also be used to fuse opposing bones in other body joints in applications such as an arthrodesis procedure.

### The Implant System with Custom Sized Components

In some embodiments of the disclosed implant system, components such as the cage or the securing element may have components customized to be sized for specific patients and for specific uses. For example, the implant system may have a cage that is custom sized from patient data to fit that specific patient and provide specific features for that patient.

Although this invention has been described in the above forms with a certain degree of particularity, it is understood that the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention which is defined in the claims and their equivalents.

We claim:

1. An orthopedic implant device comprising:
a cage;
a staple comprising a staple shaft;
a coupling mechanism comprising a coupling element configured to engage the staple shaft whereby the coupling element is configured to influence a movement of the staple shaft; and
the coupling mechanism comprising a keyway mechanism configured to constrain the movement of the staple shaft.

2. The orthopedic implant device of claim 1 wherein:
the movement of the staple shaft comprises a rotation of the staple shaft and a translation of the staple shaft;
the coupling element is configured to influence the rotation of the staple shaft and the translation of the staple shaft; and
the keyway mechanism is configured to constrain the rotation of the staple shaft and the translation of the staple shaft.

3. The orthopedic implant device of claim 1 wherein the keyway mechanism comprises:
a keyway; and
a key configured to engage the keyway to constrain the movement of the staple shaft.

4. The orthopedic implant device of claim 3 wherein:
the movement of the staple shaft is a rotation of the staple shaft; and
the key is configured to engage the keyway to constrain the rotation of the staple shaft.

5. The orthopedic implant device of claim 3 wherein:
the movement of the staple shaft is a translation of the staple shaft; and
the key is configured to engage the keyway to constrain the translation of the staple shaft.

6. The orthopedic implant device of claim 3 wherein:
the movement of the staple shaft comprises a rotation of the staple shaft and a translation of the staple shaft;
the key comprises a flexible prong coupled to the staple shaft;
the key having a rotation constraining portion configured to engage the keyway and constrain the rotation of the staple shaft;
the key having a rotation allowing portion configured to engage the keyway and allow the rotation of the staple shaft; and
the key comprises a translation stop portion configured to engage the keyway and constrain the translation of the staple shaft.

7. The orthopedic implant device of claim 3 wherein:
the movement of the staple shaft comprises a rotation of the staple shaft and a translation of the staple shaft;
the keyway comprises a through-hole in the cage;
the through-hole having a rotation constraining portion configured to engage the key and constrain the rotation of the staple shaft; and
the through-hole having a deployment position portion configured to engage the key and constrain the rotation of the staple shaft.

8. The orthopedic implant device of claim 3 wherein the implant device is configured to be implanted from a posterior approach trajectory.

9. The orthopedic implant device of claim 3 wherein the implant device further comprises a second staple.

10. The orthopedic implant device of claim 9 wherein:
the second staple comprises a second staple shaft; and
the coupling mechanism further comprises an engagement mechanism configured to influence a movement of the second staple shaft with the movement of the staple shaft.

11. The orthopedic implant device of claim 10 wherein:
the movement of the second staple shaft comprises a rotation of the second staple shaft; and
the coupling mechanism comprises an engagement portion of the staple shaft configured to engage an engagement portion of the second staple shaft whereby a rotation movement of the staple shaft influences a rotation movement of the second staple shaft.

12. The orthopedic implant device of claim 3 wherein the implant device further comprises a plate.

13. An orthopedic implant device comprising:
a cage having a proximal end and a distal end;
one or more staple positioned near the distal end of the cage;
the one or more staple configured to move relative to the cage; and
wherein the one or more staple is configured to move relative to the cage from an insertion position to an extended position.

14. The orthopedic implant device of claim 13 wherein:
the orthopedic implant device having a drive coupler configured to rotate about a drive coupler rotation axis; and
the one or more staple is configured to move to the extended position along one or more staple longitudinal axis that is non-parallel to the drive coupler rotation axis.

15. The orthopedic implant device of claim 13 wherein:
the orthopedic implant device having a drive coupler configured to rotate about a drive coupler rotation axis;
the one or more staple is configured to move to a deployed position; and
the one or more staple is configured to rotate about one or more staple rotation axis that is non-parallel to the drive coupler rotation axis to the deployed position.

16. The orthopedic implant device of claim 13 wherein:
the cage having a posterior side and an anterior side on either side of a longitudinal midline of the cage;
the posterior side of the cage is configured to be positioned in a posterior orientation to a mammalian body when the orthopedic implant device is implanted in the mammalian body;
the anterior side of the cage is configured to be positioned in an anterior orientation to the mammalian body when the orthopedic implant device is implanted in the mammalian body;
the one or more staple is positioned near the distal end of the cage;
a drive coupler positioned on the posterior side of the cage; and
the drive coupler is configured to be manipulated from the posterior side of the cage and move the one or more staple to the extended position.

17. The orthopedic implant device of claim 13 wherein:
the orthopedic implant device is configured to be inserted towards a vertebral body from an approach trajectory; and
the approach trajectory of the implant device is one of a posterior approach trajectory, a posterior lateral approach trajectory or a posterior oblique approach trajectory.

18. The orthopedic implant device of claim 13 wherein:
the orthopedic implant device is configured to be inserted towards a vertebral body from an approach trajectory; and
the one or more staple is configured to be movable about one or more axis that is non-parallel to the approach trajectory of the orthopedic implant device.

19. The orthopedic implant device of claim 18 wherein the implant device is configured to be secured to a vertebral body in an interbody fusion procedure comprising one of a posterior lumbar interbody fusion (PLIF) procedure or a transforaminal lumbar interbody fusion (TLIF) procedure.

20. The orthopedic implant device of claim 18 wherein:
the one or more staple comprises a first staple and a second staple;
the first staple having a first staple head positioned near the distal end of the cage; and
the second staple having a second staple head positioned near the proximal end of the cage.

21. The orthopedic implant device of claim 20 wherein the first staple and the second staple are coupled whereby the first staple and the second staple are configured to rotate together.

22. The orthopedic implant device of claim 20 wherein the first staple head and the second staple head are each positioned within an outer wall of the cage.

23. The orthopedic implant device of claim 20 wherein the first staple head and the second staple head are each positioned outside of an outer wall of the cage.

24. The orthopedic implant device of claim 13 further comprising a keyway mechanism configured to influence a movement of the one or more staple.

25. The orthopedic implant device of claim 24 wherein the keyway mechanism comprises:
a keyway configured to influence the movement of the one or more staple; and
a key configured to engage the keyway to constrain the movement of the one or more staple.

26. A method to secure an implant device to a vertebral body, the method comprising:
providing access to a vertebral body from an access portal positioned posterior to the vertebral body;
performing an osteotomy through the vertebral body to create an osteotomy space between a first bone portion and a second bone portion;
providing an implant device comprising a cage and one or more staple;
positioning the implant device in the osteotomy space created within the vertebral body from a posterior approach trajectory;
the cage movably coupled to the one or more staple; and
moving the one or more staple to engage the first bone portion and the second bone portion of the vertebral body whereby the one or more staple secures the implant device to the first bone portion and the second bone portion of the vertebral body.

27. The method of claim 26 wherein:
the cage is movably coupled to the one or more staple with a coupling mechanism;
the coupling mechanism comprising one or more drive coupler configured to move the one or more staple;
the one or more drive coupler is configured to be engaged by one or more drive rod from the access portal positioned posterior to the vertebral body; and
the step of moving the one or more staple to engage the first bone portion and the second bone portion of the vertebral body comprises:
engaging the one or more drive coupler with the one or more drive rod to move the one or more staple to a deployed position, and
engaging the one or more drive coupler with the one or more drive rod to move the one or more staple from the deployed position to a stabilized position whereby the cage is secured to the first bone portion and the second bone portion of the vertebral body.

28. The method of claim 26 wherein:
the one or more staple comprises a first staple and a second staple;
the first staple having a first staple head positioned proximal to a distal end of the cage; and
the second staple having a second staple head positioned proximal to a proximal end of the cage;
the first staple head and the second staple head are each positioned outside of an outer wall of the cage; and
the first staple head and the second staple head are configured to engage an outer wall of both the first bone portion and the second bone portion of the vertebral body when the first staple and the second staple are in a stabilized position.

29. The method of claim 26 wherein:
the one or more staple comprises a first staple and a second staple;
the first staple having a first staple head positioned near a distal end of the cage; and
the second staple having a second staple head positioned near a proximal end of the cage;
the first staple head and the second staple head are each positioned within an outer wall of the cage; and
the first staple head and the second staple head are configured to engage both the first bone portion and the second bone portion of the vertebral body when the first staple and the second staple are in a stabilized position.

30. The method of claim 26 wherein:
the staple comprises a first staple and a second staple;
the cage comprises a first cage section coupled to the first staple and a second cage section coupled to the second staple;
the first staple having a first staple head positioned near a distal end of the cage whereby the first staple head engages both the first bone portion and the second bone portion when the implant device is in a stabilized position; and
the second staple having a second staple head positioned near a proximal end of the cage whereby the second staple head engages both the first bone portion and the second bone portion when the implant device is in a stabilized position.
